SEQUENCE LISTING (43 sequences)

SEQ ID NO: 1 - DNA, 20 bp, synthetic construct
  source 1..20: mol_type = other DNA
cgccatgaacagcatgactg

SEQ ID NO: 2 - DNA, 20 bp, synthetic construct
  source 1..20: mol_type = other DNA
catgaacatgtcgtcgtacg

SEQ ID NO: 3 - DNA, 20 bp, synthetic construct
  source 1..20: mol_type = other DNA
gcgactggaacagctactac

SEQ ID NO: 4 - DNA, 20 bp, synthetic construct
  source 1..20: mol_type = other DNA
gcactgcaatactcgcctta

SEQ ID NO: 5 - DNA, 20 bp, synthetic construct
  source 1..20: mol_type = other DNA
tccgactggagcagctacta

SEQ ID NO: 6 - DNA, 20 bp, synthetic construct
  source 1..20: mol_type = other DNA
cggctacggttcccccatgc

SEQ ID NO: 7 - DNA, 20 bp, synthetic construct
  source 1..20: mol_type = other DNA
aaaacaatctgaggagaaca

SEQ ID NO: 8 - DNA, 200 bp, synthetic construct
  source 1..200: mol_type = other DNA
tgcctatgatttatgtatttgtttagtcaatagtctaatgtaaatgatgtaattaattatagatggtggtgtcaggtcatttgtgtaaacaatctgaggtaaacaagggctctgtttacttcatgacagatgcaggggggtggggggctgagttgagggaattccaggggaactttttcacgtgtgaatggcggctggga

SEQ ID NO: 9 - DNA, 55 bp, synthetic construct
  source 1..55: mol_type = other DNA
tgaacaagatgaagagcaccttcaagagaggtgctcttcatcttgttcttttttc

SEQ ID NO: 10 - DNA, 58 bp, synthetic construct
  source 1..58: mol_type = other DNA
tcgagaaaaaagaacaagatgaagagcacctctcttgaaggtgctcttcatcttgtca

SEQ ID NO: 11 - DNA, 55 bp, synthetic construct
  source 1..55: mol_type = other DNA
tggagttcctattcaacaagttcaagagacttgttgaataggaactccttttttc

SEQ ID NO: 12 - DNA, 58 bp, synthetic construct
  source 1..58: mol_type = other DNA
tcgagaaaaaaggagttcctattcaacaagtctcttgaacttgttgaataggaactca

SEQ ID NO: 13 - DNA, 20 bp, synthetic construct
  source 1..20: mol_type = other DNA
aaaggggaagggagcacatg

SEQ ID NO: 14 - DNA, 21 bp, synthetic construct
  source 1..21: mol_type = other DNA
tcctgcacactgtctactgat

SEQ ID NO: 15 - DNA, 22 bp, synthetic construct
  source 1..22: mol_type = other DNA
gctgggatctcaatttgcatgt

SEQ ID NO: 16 - DNA, 20 bp, synthetic construct
  source 1..20: mol_type = other DNA
agggagcagctggttcttta

SEQ ID NO: 17 - DNA, 23 bp, synthetic construct
  source 1..23: mol_type = other DNA
aattatagatggtggtgtcaggt

SEQ ID NO: 18 - DNA, 20 bp, synthetic construct
  source 1..20: mol_type = other DNA
aaattgagatcccagccgcc

SEQ ID NO: 19 - DNA, 21 bp, synthetic construct
  source 1..21: mol_type = other DNA
gatggcacactgacagaagat

SEQ ID NO: 20 - DNA, 21 bp, synthetic construct
  source 1..21: mol_type = other DNA
gcagagtcaataggtgcagac

SEQ ID NO: 21 - DNA, 20 bp, synthetic construct
  source 1..20: mol_type = other DNA
tcaaaggctcatctttgcag

SEQ ID NO: 22 - DNA, 20 bp, synthetic construct
  source 1..20: mol_type = other DNA
aaagctggactggtgaatgc

SEQ ID NO: 23 - DNA, 21 bp, synthetic construct
  source 1..21: mol_type = other DNA
aagtctaccaaagctcacgcg

SEQ ID NO: 24 - DNA, 15 bp, synthetic construct
  source 1..15: mol_type = other DNA
gtaggcgccgcctgc

SEQ ID NO: 25 - DNA, 20 bp, synthetic construct
  source 1..20: mol_type = other DNA
ctggcctgtacccctcatca

SEQ ID NO: 26 - DNA, 20 bp, synthetic construct
  source 1..20: mol_type = other DNA
cttcccgtctttgtccaaca

SEQ ID NO: 27 - DNA, 20 bp, synthetic construct
  source 1..20: mol_type = other DNA
gaagatggaagggcatgaaa

SEQ ID NO: 28 - DNA, 20 bp, synthetic construct
  source 1..20: mol_type = other DNA
gcctgagttcatgttgctga

SEQ ID NO: 29 - DNA, 18 bp, synthetic construct
  source 1..18: mol_type = other DNA
gggagcggtgaagatgga

SEQ ID NO: 30 - DNA, 22 bp, synthetic construct
  source 1..22: mol_type = other DNA
tcatgttgctcacggaggagta

SEQ ID NO: 31 - DNA, 24 bp, synthetic construct
  source 1..24: mol_type = other DNA
aagcatttactttgtggctggatt

SEQ ID NO: 32 - DNA, 25 bp, synthetic construct
  source 1..25: mol_type = other DNA
tgatctggatttctcctctgtgtct

SEQ ID NO: 33 - DNA, 20 bp, synthetic construct
  source 1..20: mol_type = other DNA
tgtgcacaggagccaagagt

SEQ ID NO: 34 - DNA, 21 bp, synthetic construct
  source 1..21: mol_type = other DNA
attttcttgctgccagtctgg

SEQ ID NO: 35 - DNA, 48 bp, synthetic construct
  source 1..48: mol_type = other DNA
ggtcattggtgaaaacaatctgaggagaacaagggctctgtttttttc

SEQ ID NO: 36 - DNA, 48 bp, synthetic construct
  source 1..48: mol_type = other DNA
gaaaaaaacagagcccttgttctcctcagattgttttcaccaatgacc

SEQ ID NO: 37 - DNA, 20 bp, synthetic construct
  source 1..20: mol_type = other DNA
ccgcagcatgctgttcatgg

SEQ ID NO: 38 - DNA, 21 bp, synthetic construct
  source 1..21: mol_type = other DNA
agcgcccacgttacgacgatg

SEQ ID NO: 39 - DNA, 23 bp, synthetic construct
  source 1..23: mol_type = other DNA
cgcagatgctgttcatggcgccc

SEQ ID NO: 40 - DNA, 11 bp, synthetic construct
  source 1..11: mol_type = other DNA
tactaccggct

SEQ ID NO: 41 - DNA, 11 bp, synthetic construct
  source 1..11: mol_type = other DNA
  modified_base 7: mod_base = OTHER; note = inverted nucleotide
  modified_base 8: mod_base = OTHER; note = inverted nucleotide
  modified_base 9: mod_base = OTHER; note = inverted nucleotide
  modified_base 10: mod_base = OTHER; note = inverted nucleotide
  modified_base 11: mod_base = OTHER; note = inverted nucleotide
tactacgacaa

SEQ ID NO: 42 - DNA, 14 bp, synthetic construct
  source 1..14: mol_type = other DNA
agcgcccacgtatg

SEQ ID NO: 43 - DNA, 12 bp, synthetic construct
  source 1..12: mol_type = other DNA
  modified_base 7: mod_base = OTHER; note = inverted nucleotide
  modified_base 8: mod_base = OTHER; note = inverted nucleotide
  modified_base 9: mod_base = OTHER; note = inverted nucleotide
  modified_base 10: mod_base = OTHER; note = inverted nucleotide
  modified_base 11: mod_base = OTHER; note = inverted nucleotide
  modified_base 12: mod_base = OTHER; note = inverted nucleotide
tactaccccatc

(19) United States
(12) Patent Application Publication
(10) Pub. No.: US 2025/0101385 A1
(43) Pub. Date: Mar. 27, 2025

(54) GENE EDITING TO IMPROVE PANCREATIC BETA CELL YIELD IN DIRECTED DIFFERENTIATION OF HUMAN PLURIPOTENT STEM CELLS

(71) Applicant: The Regents of the University of California, Oakland, CA (US)

(72) Inventors: Maike SANDER, La Jolla, CA (US); Ryan GEUSZ, La Jolla, CA (US)

(73) Assignee: The Regents of the University of California, Oakland, CA (US)

(21) Appl. No.: 18/728,977

(22) PCT Filed: Jan. 4, 2023
(86) PCT No.: PCT/US2023/060090

§ 371 Date: Jul. 15, 2024

Related U.S. Application Data
(60) Provisional application No. 63/300,144, filed on Jan. 17, 2022.

(51) Int. Cl. C12N 5/071; A61K 35/39; C07K 14/47; C07K 14/62; C12N 9/22; C12N 15/11

(52) U.S. Cl. CPC C12N 5/0676; A61K 35/39; C07K 14/4702; C07K 14/62; C12N 9/22; C12N 15/111; C12N 2310/20; C12N 2506/45

(57) ABSTRACT

The present disclosure addresses cell type heterogeneity during differentiation of human pluripotent stem cells into islet cells by shifting cell identity from alpha cells to beta cells. More specially, the present disclosure provides a clonal human pluripotent stem cell line that broadly expresses the gene NKX6.1 across cells at the pancreatic progenitor cell stage of in vitro differentiation, which yields fewer alpha cells and more beta cells during directed differentiation to islet cells. The present disclosure further provides a method of improving the yield of insulin-producing cells produced from pluripotent stem cells for transplantation into patients with diabetes.

### CROSS-REFERENCE TO RELATED APPLICATION

This application claims the benefit of U.S. Provisional Application No. 63/300,144, filed on Jan. 17, 2022, the entire content of which is incorporated herein by reference.

### STATEMENT REGARDING FEDERALLY SPONSORED RESEARCH OR DEVELOPMENT

This invention was made with government support under DK068471, and DK078803 awarded by the National Institutes of Health. The government has certain rights in the invention.

### BACKGROUND OF THE INVENTION

The pancreas, liver, and lung develop from the foregut endoderm in response to local signaling cues that specify lineage identity by inducing organ-specific gene expression. The competence of organ lineage precursors to activate lineage-specific genes in response to inductive signals is acquired during endoderm development^{1, 2}. Coincident with the acquisition of competence, the transcription factors (TFs) FOXA1 and FOXA2 (henceforth abbreviated FOXA1/2) are recruited to enhancers of foregut-derived organ lineages, leading to a gain in chromatin accessibility and H3K4me1 deposition^{1, 3, 4}, a phenomenon referred to as enhancer priming. Thus, current evidence suggests that FOXA1/2's role in endodermal organ development is to render foregut endoderm competent to activate organ-specific genes by broadly priming pancreas-, liver-, and lung-specific enhancers before organ-inductive signals trigger enhancer activation. Consistent with this model, studies in model organisms and human pluripotent stem cell (hPSC)-based differentiation systems have shown a requirement for FOXA1/2 in pancreas, liver, and lung development, with the two FOXA TFs functioning in a partially or fully redundant manner^{3, 4, 5, 6}. However, whether chromatin priming is the only mechanism by which FOXA TFs control endodermal organ development is unknown.

The mechanisms by which FOXA TFs engage with and open chromatin have been the subject of debate. In vitro experiments have shown that FOXA TFs possess pioneering activity, which refers to the specific ability of a TF to engage target sites on nucleosomal DNA and to remodel such regions to increase chromatin accessibility^{7, 8, 9}. Through their chromatin remodeling activity, FOXA TFs facilitate subsequent binding of other TFs and co-factors that further modify chromatin state and initiate gene expression^{9, 10, 11, 12, 13, 14}. However, despite their ability to access target sites in closed chromatin in vitro, binding site selection of FOXA and other pioneer TFs in cellular contexts has been shown to depend on additional features, such as the local chromatin landscape¹⁵, presence of cooperative binding partners^{16, 17}, and strength of the binding motif^{17, 18, 19}. For example, steroid receptor activation in breast cancer cell lines induces FOXA1 recruitment to sites with degenerate FOXA1-binding motifs^{18, 20}, exemplifying heterogeneity in FOXA target site engagement. The determinants that underlie FOXA-binding site selection and FOXA-mediated enhancer priming during cellular transitions of development remain to be explored.

### SUMMARY OF THE INVENTION

The present disclosure provides a method of improving the yield of insulin-producing cells produced from pluripotent stem cells for transplantation into patients with diabetes. More specially, the present disclosure provides a clonal human pluripotent stem cell line that broadly expresses the gene NKX6.1 across cells at the pancreatic progenitor cell stage of in vitro differentiation, which yields fewer alpha cells and more beta cells during directed differentiation to islet cells. Therefore, the present disclosure provides a method of treating diabetes using the beta cells produced from the method disclosed in the present disclosure.

In certain embodiments, the present disclosure provides specific mechanisms that underlie the regulation of endodermal organ development by FOXA TFs. In certain embodiments, FOXA1/2 genomic association with pancreas-specific enhancers was mapped throughout a time course of hPSC differentiation into pancreas. Surprisingly, only a minority of pancreas-specific enhancers are FOXA1/2-bound prior to lineage induction and exhibit priming, whereas the majority engage FOXA1/2 concomitant with pancreas induction. Compared to unprimed enhancers, primed enhancers contain DNA sequences more closely matching FOXA consensus motifs and harbor additional sequence motifs for signal-dependent TFs. By contrast, unprimed enhancers contain degenerate and fewer FOXA motifs, are enriched for motifs of lineage-specific TFs, and depend on the pancreas-specific TF PDX1 for FOXA1/2 recruitment.

Further, in certain embodiments, the present disclosure provides that CRISPR/Cas9-mediated optimization of FOXA motifs in an unprimed enhancer near the pancreatic TF NKX6.1 is sufficient to redefine patterns of FOXA binding and to broaden NKX6.1 expression within the pancreatic progenitor domain, suggesting that FOXA motif strength is relevant for fine-tuning developmental gene expression. In-depth analysis of FOXA binding during hPSC differentiation toward hepatocytes and lung alveolospheres revealed similar patterns of FOXA binding and sequence logic at FOXA-bound enhancers. These findings show that FOXA1/2 regulate foregut organ development through two distinct and complementary mechanisms: priming of a small subset of organ-specific enhancers before lineage induction and activation of a larger cohort of enhancers through cooperative binding with organ lineage-specific TFs.

In view of these finding, the present disclosure provides that priming of a small enhancer subset permits precise spatial and temporal regulation of organ induction by lineage-inductive signals, whereas cooperative FOXA binding with lineage-specific TFs ensures cell type specificity of gene expression, providing a safeguard against broad activation of alternative lineage programs during developmental transitions.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the drawings and supplemental drawings, tables, data and description attached as Appendix I, which is incorporated by reference by its entity. The components in the drawings and supplemental drawings are not necessarily to scale, emphasis instead being placed upon clearly illustrating the principles of the present disclosure. Moreover, in the drawings, like reference numerals designate corresponding parts throughout the several views.

FIGS. 1a-1g. Partially redundant requirement for FOXA1 and FOXA2 in pancreatic lineage induction. FIG. 1a. Schematic of stepwise pancreatic differentiation protocol from hESCs (ES): definitive endoderm (DE), primitive gut tube (GT), early pancreatic progenitor cells (PP1), and late pancreatic progenitor cells (PP2), with indicated genetic modifications in ES. RA, retinoic acid; KGF, keratinocyte growth factor. FIG. 1b. qPCR analysis of PDX1 and NKX6.1 (left), immunofluorescent staining (middle), and flow cytometry quantification of PDX1⁺ and NKX6.1⁺ cells (right) in control, FOXA1^{-/-} and FOXA2^{-/-} PP2 cells (qPCR: P=0.493, 0.590, 3.12×10⁻³, and <1.00×10⁻⁶ for PDX1 and NKX6.1 in control compared to FOXA1^{-/-} and FOXA2^{-/-} PP2 cells, respectively; flow cytometry: P=1.15×10⁻² and 7.00×10⁻⁴ in control compared to FOXA1^{-/-} and FOXA2^{-/-} PP2 cells, respectively). FIG. 1c. qPCR analysis of FOXA1 and FOXA2 in control, FOXA1^{-/-} and FOXA2^{-/-} PP2 cells (P=<1.00×10⁻⁶ and 0.700 for FOXA1 and FOXA2 in control compared to FOXA1^{-/-} and FOXA2^{-/-} PP2 cells, respectively). FIG. 1d Immunofluorescent staining (left) and flow cytometry quantification (right) of PDX1⁺ and NKX6.1⁺ cells in control and FOXA1/2^{-/-} PP2 cells. (P=2.6×10⁻³ in control compared to FOXA1/2^{-/-} PP2 cells). FIG. 1e. mRNA expression levels of pancreatic transcription factors determined by RNA-seq in control and FOXA1/2^{-/-} PP2 cells (n=4 independent differentiations; P adj.=1.08×10⁻⁴², 2.56×10⁻¹², 4.93×10⁻²⁰, 1.00×10⁻⁴⁹, and 2.82×10⁻⁴ for PDX1, NKX6.1, PROX1, PTF1A, and SOX9, respectively; DESeq2; FPKM, fragments per kilobase per million fragments mapped). FIG. 1f. Enriched gene ontology terms of 2833 downregulated genes (≥2-fold decrease, P adj.<0.05) in FOXA1/2^{-/-} compared to control PP2 cells. FIG. 1g. Principal component analysis showing variance in total normalized transcriptome between control and FOXA1/2^{-/-} cells in GT and PP2. Each point represents one biological replicate. For all qPCR, each plotted point represents the average of three technical replicates. For all immunofluorescence, representative images are shown from n≥2 independent differentiations. Scale bars, 50 μm. For all flow cytometry analyses, representative plots are shown from n=3 independent differentiations.

FIGS. 2a-2e. Two distinct temporal patterns of FOXA1 and FOXA2 binding to pancreatic enhancers. FIGS. 2a and 2b. Heatmaps showing density of FOXA1 and FOXA2 ChIP-seq reads (FIG. 2a) and H3K27ac ChIP-seq reads (FIG. 2b) at pancreatic enhancers in GT and PP2. Heatmaps are centered on FOXA1, FOXA2, and H3K27ac peaks, respectively, and span 5 kb. Pancreatic enhancers are classified based on temporal pattern of FOXA1 and FOXA2 occupancy. FIG. 2c. Box plots of H3K27ac ChIP-seq counts at class I and class II pancreatic enhancers in control and FOXA1/2^{-/-} GT and PP2 cells (P=<2.2×10⁻¹⁶, <2.2×10⁻¹⁶, 0.009, and <2.2×10⁻¹⁶ for control versus FOXA1/2^{-/-} at class I enhancers in GT, class I enhancers in PP2, class II enhancers in GT, and class II enhancers in PP2, respectively; Wilcoxon rank sum test, 2-sided). Plots are centered on median, with box encompassing 25th-75th percentile and whiskers extending up to 1.5 interquartile range. FIG. 2d. Genome browser snapshots showing FOXA1, FOXA2, and H3K27ac ChIP-seq signal at class I pancreatic enhancers near PDX1 and HNF1B and class II pancreatic enhancers near NKX6.1 and MNX1 in GT and PP2. Approximate distance between enhancer and gene body is indicated. FIG. 2e. Schematic illustrating the identified pattern of FOXA1/2 occupancy at pancreatic enhancers. All ChIP-seq experiments, n=2 replicates from independent differentiations.

FIGS. 3a-3f. Class I and class II pancreatic enhancers largely map to distinct gene regulatory elements. FIG. 3a. Tag density plots for class I and class II pancreatic enhancers displaying ATAC-seq (top) and H3K4me1 ChIP-seq (bottom) read density in GT and PP2. Plots are centered on FOXA1/2 peaks and span 5 kb. FIG. 3b. Box plots of ATAC-seq (top) and H3K4me1 ChIP-seq (bottom) counts at class I and class II pancreatic enhancers in GT and PP2 for control and FOXA1/2^{-/-} cells (P=<2.2×10⁻¹⁶, <2.2×10⁻¹⁶, 0.01, and <2.2×10⁻¹⁶ for control versus FOXA1/2^{-/-} of ATAC-seq signal at class I in GT, class I in PP2, class II in GT, and class II in PP2, respectively. P=<2.2×10⁻¹⁶, 0.01, 0.02, and 0.01 for control versus FOXA1/2^{-/-} of H3K4me1 signal at class I in GT, class I in PP2, class II in GT, and class II in PP2, respectively; Wilcoxon rank sum test, 2-sided). Plots are centered on median, with box encompassing 25th-75th percentile and whiskers extending up to 1.5 interquartile range. FIG. 3c. Percentage of FOXA1- and/or FOXA2-bound pancreatic super-enhancers (SEs) in PP2 containing only class I, only class II, or both class I and class II enhancers. FIG. 3d. Percentage of chromatin loop anchors in PP2 containing only class I, only class II, or both class I and class II enhancers. FIG. 3e. Percentage of genes associated with only class I, only class II, or both class I and class II enhancers. Target genes were assigned to enhancers based on nearest TSS of expressed genes (fragments per kilobase per million fragments mapped (FPKM)≥1) in PP2. FIG. 3f. Schematic illustrating FOXA1/2 occupancy, chromatin accessibility, and presence of H3K4me1 and H3K27ac at class I and class II enhancers in GT and PP2. All ChIP-seq and ATAC-seq experiments, n=2 replicates from independent differentiations.

FIGS. 4a-4e. FOXA1/2-binding sites at class I and class II pancreatic enhancers differ in DNA sequence. FIG. 4a. Enriched de novo transcription factor (TF)-binding motifs at class I against a background of class II pancreatic enhancers and vice versa. Fisher's exact test, 1-sided, corrected for multiple comparisons. FIG. 4b. Percentage of class I and class II enhancers overlapping RXR ChIP-seq peaks in PP1; GATA4 and GATA6 ChIP-seq peaks in GT; and HNF6, PDX1, and SOX9 ChIP-seq peaks (within 100 bp from peak) in PP2 (P=8.27×10⁻¹⁴, <2.2×10⁻¹⁶, <2.2×10⁻¹⁶, 3.52×10⁻⁴, 1.01×10⁻⁵, and 0.40 for comparisons of overlap with binding sites for RXR, GATA4, GATA6, HNF6, PDX1, and SOX9, respectively; Fisher's exact test, 2-sided). FIG. 4c. Percentage of class I and class II enhancers with at least one occurrence of selected FOXA1 and FOXA2 motifs (P=<2.2×10⁻¹⁶, <2.2×10⁻¹⁶, 1.76×10⁻¹³, 1.61×10⁻⁴, <2.2×10⁻¹⁶, and <2.2×10⁻¹⁶ for comparisons of occurrences of MA0148.1, MA0148.3, MA0148.4, MA0047.1, MA0047.2, and MA0047.3, respectively. Fisher's exact test, 2-sided). FIG. 4d. Probability (motif occurrence per base pair) of FOXA1 (MA0148.3) and FOXA2 (MA0047.2) motifs relative to ATAC-seq peak summits at class I (solid line) and class II (dashed line) enhancers. ATAC-seq peak summits at class I enhancers are enriched for occurrences of MA0148.3 (P=2.1×10⁻¹⁴; Fisher's exact test, 1-sided) and MA0047.2 (P=6.8×10⁻¹⁴) compared to summits at class II enhancers. FIG. 4e. Percentage of class I and class II enhancers containing FOXA TF ATAC-seq footprints in PP2 (P=1.01×10⁻¹⁰ for comparison of class I and class II enhancers; Fisher's exact test, 2-sided). All ChIP-seq experiments, n=2 replicates from independent differentiations.

FIGS. 5a-5d. FOXA1/2 binding at class II enhancers is dependent on PDX1. FIG. 5a. Schematic of experimental design for PDX1 knock-down in hESCs and subsequent differentiation into PP2 stage pancreatic progenitors. FIG. 5b. Heatmap showing density of FOXA1 and FOXA2 ChIP-seq reads at PDX1-bound class I and class II pancreatic enhancers in hESCs transduced with scrambled control (SCRAM) or PDX1 shRNA (shPDX1) in PP2. Heatmap is centered on FOXA1 and FOXA2 peaks, respectively, and spans 5 kb. FIG. 5c. Genome browser snapshots showing PDX1, FOXA1, and FOXA2 ChIP-seq, ATAC-seq, and H3K27ac ChIP-seq signal at class I enhancers near PDX1 and HNF1B and class II enhancers near NKX6.1 and MNX1 in PP2. Approximate distance between enhancer and gene body is indicated. FIG. 5d. Schematic illustrating distinct modes of FOXA TF recruitment at class I and class II pancreatic enhancers. FOXA1/2 recruitment depends on the lineage-determining TF PDX1 at class II enhancers. Both enhancer classes require PDX1 for activation. All ChIP-seq and ATAC-seq experiments, n=2 replicates from independent differentiations.

FIGS. 6a-6f. Optimization of FOXA-binding motifs at an NKX6.1 enhancer redefines patterns of FOXA association and gene expression. FIG. 6a. Schematic illustrating base editing strategy at NKX6.1 enhancer via CRISPR-Cas9. Degenerate FOXA-binding motifs and base edits are indicated in red. Figure discloses SEQ ID NOS 35-36, respectively, in order of appearance. FIG. 6b. ChIP-qPCR comparing FOXA1, FOXA2, H3K4me1, and H3K27ac ChIP-seq signal at the NKX6.1 enhancer in control and motif optimized hESC lines at GT stage. Plots show two independent primer pairs for NKX6.1 enhancer and one primer pair for a negative control region; n=3 technical replicates. FIG. 6c. UMAP representation of single-cell RNA-seq data from both control and motif optimized PP2 cells (integrated) and dot plot showing expression of marker genes in each population (bottom). NKX6.1 expression across populations in control and motif optimized cell lines (right). FIG. 6d. Volcano plot comparing genes co-expressed with NKX6.1 in motif optimized compared to control PP2 cells. Wilcoxon rank sum test, 2-sided, corrected for multiple comparisons. FIG. 6e. Representative flow cytometry analysis for PDX1 and NKX6.1, mean fluorescence intensity (MFI) of PDX1 signal in NKX6.1⁺ cells, and quantification of PDX1⁺ and NKX6.1⁺ cells in control and motif optimized PP2 cells (n=3 independent differentiations; P<1.0×10⁻⁴; student's t-test, 2-sided). Bar graph shows mean±S.E.M. FIG. 6f. Schematic illustrating temporal patterns of FOXA recruitment and NKX6.1 expression at the PP2 stage in cells with degenerate and optimized FOXA motifs at the NKX6.1 enhancer.

FIGS. 7a-7h. Class I and class II enhancers can be distinguished in liver and lung development. FIGS. 7a and 7b Schematic of stepwise differentiation of hESCs to hepatic progenitors (HP) (FIG. 7a) and induced human pluripotent stem cells (iPSC) into alveolosphere organoids (ALV) (FIG. 7b). AFG, anteriorized foregut. Select growth factors for hepatic (FIG. 7a) and alveolar (FIG. 7b) lineage induction are indicated. FGF2, fibroblast growth factor 2; BMP4, bone morphogenic protein 4; CHIR, CHIR99021; RA, retinoic acid. FIG. 7c. Heatmap showing density of FOXA1 and FOXA2 ChIP-seq reads at hepatic enhancers in GT and HP. Heatmap is centered on FOXA1 and FOXA2 peaks, respectively, and spans 5 kb. Hepatic enhancers are classified based on temporal pattern of FOXA1 and FOXA2 occupancy. FIG. 7d. Heatmap showing density of FOXA1 ChIP-seq reads at alveolar enhancers in AFG and ALV. Heatmap is centered on FOXA1 peaks and spans 5 kb. Alveolar enhancers are classified based on temporal pattern of FOXA1 occupancy. FIG. 7e. Genome browser snapshots showing FOXA1 and FOXA2 ChIP-seq signal at a class I hepatic enhancer near AAT and a class II hepatic enhancer near CEBPA in GT and HP. FIG. 7f. Genome browser snapshots showing FOXA1 ChIP-seq signal at a class I alveolar enhancer near SOX2 and a class II alveolar enhancer near NKX2.1 in AFG and ALV. FIGS. 7g and 7h Enriched de novo transcription factor (TF)-binding motifs at class I against a background of class II enhancers and vice versa for hepatic (FIG. 7g) and alveolar enhancers (FIG. 7h). Fisher's exact test, 1-sided, corrected for multiple comparisons. All ChIP-seq experiments, n=2 replicates from independent differentiations.

FIGS. 8a-8d. Recruitment of FOXA1/2 to class II enhancers is lineage-specific. FIG. 8a. Heatmap showing enrichment of known binding motifs for lineage-determining transcription factors at pancreatic, hepatic, and alveolar class I and class II enhancers. Class I and class II enhancers of each lineage were compared against a background of class I and class II enhancers, respectively, of all other lineages. Fisher's exact test, 1-sided, corrected for multiple comparisons. FIG. 8b. Percentage of pancreatic, hepatic, and alveolar class I and class II enhancers overlapping FOXA1/2 ChIP-seq peaks (within 100 bp from peak) in PP2 (pancreas), HP (liver) and ALV (lung). For ALV only FOXA1 peaks were considered. P=1, <2.2×10⁻¹⁶, <2.2×10⁻¹⁶, <2.2×10⁻¹⁶, 1, <2.2×10⁻¹⁶, 5.86×10⁻¹¹, 3.08×10⁻⁵, and 1 for comparisons of FOXA occupancy at class I and class II pancreatic, hepatic, and alveolar enhancers at PP2, HP, and ALV stage cells, respectively; Fisher's exact test, 2-sided). FIG. 8c. Genome browser snapshots showing FOXA1/2 ChIP-seq signal across endodermal lineages at example pancreatic, hepatic, and alveolar class I and class II enhancers. Approximate distance between enhancer and gene body is indicated. FIG. 8d. Schematic showing differential recruitment of FOXA TFs to endodermal organ class I and class II enhancers during endoderm development. LDTF, lineage-determining transcription factor. All ChIP-seq experiments, n=2 replicates from independent differentiations.

FIGS. 9a-9g. FOXA1 and FOXA2 promote pancreatic lineage induction. FIG. 9a. Heatmap showing mRNA expression levels of FOXA1 and FOXA2 determined by RNA-seq during pancreatic differentiation of hESCs. FPKM, Fragments per kilobase per million fragments mapped. FIG. 9b. Immunofluorescent staining of FOXA1 and FOXA2 in GT and PP2. FIG. 9c. Schematic of frameshift mutation in FOXA1^{-/-} hESCs (left) and immunofluorescent staining (right) of FOXA1 in PP2. Figure discloses “ccgcagcatgctgttcatgg” as SEQ ID NO: 37. FIG. 9d. Schematic of frameshift mutation in FOXA2^{-/-} hESCs (left) and immunofluorescent staining (right) of FOXA2 in PP2. Figure discloses “agcgcccacgttacgacgatg” as SEQ ID NO: 38. FIG. 9e. Schematic of frameshift mutations and exon deletions in FOXA1/2^{-/-} hESCs (left) and immunofluorescent staining (right) of FOXA1 and FOXA2 in PP2. Figure discloses SEQ ID NOS 39 (top left), 40-41 (top right), 42 and 38 (bottom left), and 40, and 43 (bottom right), respectively, in order of appearance. FIGS. 9f and 9g. mRNA expression levels determined by RNA-seq (left), immunofluorescent staining (right), and flow cytometry quantification (bottom) in control and FOXA1/2^{-/-} DE (FIG. 9f) and GT (FIG. 9g) cells (n=4 and n=3 independent differentiations for RNA-seq and flow cytometry, respectively; qPCR: P adj.=0.116 and 0.104 for SOX17 and GATA4, respectively, in FIG. 9f and 0.014 and 0.061 for HNF1B and HNF4A, respectively, in FIG. 9g; DESeq2; flow cytometry: P=0.116 in control compared to FOXA1/2^{-/-} DE cells in FIG. 9f and 4.5×10⁻³ in control compared to FOXA1/2^{-/-} GT cells in FIG. 9g; student's t-test, 2 sided). Bar graphs show mean±S.E.M. FSC-A, forward scatter area. For all immunofluorescence, representative images are shown from n≥2 independent differentiations; scale bars, 50 μm.

FIG. 10. Gating strategy for FACS analysis. Cells were gated to identify live cells (FSCA vs SSC-A) and singlets (FSC-A vs FSC-W, SSC-A vs SSC-W). Isotype controls were performed using antibodies against IgG conjugated to corresponding fluorophores.

FIGS. 11a-11g. FOXA1 and FOXA2 both bind to pancreas-specific enhancers. FIG. 11a. Pearson correlation between FOXA1 and FOXA2 ChIP-seq signal at FOXA1 and FOXA2 peaks in GT and PP2. FIG. 11b. Percentage of FOXA1 and FOXA2 peaks located proximal (≤2.5 kb) or distal (>2.5 kb) to nearest annotated TSS. FIG. 11c. Principal component analysis showing variance in distal (>2.5 kb from TSS) H3K27ac signal between control and FOXA1/2^{-/-} cells in GT and PP2. Each point represents one biological replicate. FIG. 11d. Volcano plot showing identification of pancreatic enhancers based on increase in H3K27ac signal from GT to PP2 (≥2-fold increase, P adj.<0.05 at sites >2.5 kb from TSS; DESeq2). Enriched gene ontology terms of genes linked to pancreatic enhancers using GREAT. FIG. 11e. Box plots of H3K27ac ChIP-seq counts at pancreatic enhancers. FIG. 11f. Enrichment of pancreatic enhancers for FOXA1 or FOXA2 peaks compared to random genomic regions at GT and PP2 (P<0.0001 and P<0.0001, respectively; permutation test). Pancreatic enhancers are enriched for FOXA1/2 peaks at PP2 compared to GT (P<2.2×10⁻¹⁶, Fisher's exact test, 2-sided). FIG. 11g. Box plots of H3K27ac ChIP-seq counts at class III pancreatic enhancers in control and FOXA1/2^{-/-} GT and PP2 cells (P=1.47×10⁻⁵ and 3.17×10⁻⁶ for control versus FOXA1/2^{-/-} at class III enhancers in GT and PP2, respectively; Wilcoxon rank sum test, 2-sided). All box plots are centered on median, with box encompassing 25th-75th percentile and whiskers extending up to 1.5 interquartile range. All ChIP-seq experiments, n=2 replicates from independent differentiations.

FIGS. 12a-12g. Characterization of class I and class II pancreatic enhancers. FIG. 12a. Box plots of ATAC-seq and H3K4me1 ChIP-seq counts at class I and class II enhancers in GT and PP2 (P=<2.2×10⁻¹⁶, 1.11×10⁻¹⁴, <2.2×10⁻¹⁶, and 1.07×10⁻⁷ for comparisons of ATAC-seq signal in GT and PP2 and comparisons of H3K4me1 ChIP-seq signal in GT and PP2, respectively; Wilcoxon rank sum test, 2-sided). FIG. 12b. Genome browser snapshots showing FOXA1, FOXA2, H3K4me1 ChIP-seq, and ATAC-seq signal at class I enhancer near PDX1 and class II enhancer near NKX6.1 in GT and PP2 cells. Approximate distance between enhancer and gene body is indicated. FIG. 12c. Percentage of class I and class II enhancers overlapping H3K4me1 ChIP-seq peaks (within 1 kb from peak) in GT. FIG. 12d. Identification of pancreatic super-enhancers by ranking 2574 pancreatic enhancers based on H3K27ac ChIP-seq signal in PP2. FIG. 12e. Percentage of pancreatic super-enhancers containing FOXA1 and/or FOXA2 ChIP-seq peaks in PP2. FIG. 12f. Box plots of mRNA levels (FPKM, fragments per kilobase per million fragments mapped) in control and FOXA1/2^{-/-} GT and PP2 cells for genes linked to pancreatic class I and class II enhancers (n=4 independent differentiations; P=0.182, 4.82×10⁻³, 0.067, and 1.21×10⁻³ for control versus FOXA1/2 of class I genes in GT, class I genes in PP2, class II genes in GT, and class II genes in PP2, respectively; Wilcoxon rank sum test, 2-sided). FIG. 12g. Gene ontology terms enriched in genes associated with class I compared to class II enhancers. All box plots are centered on median, with box encompassing 25th-75th percentile and whiskers extending up to 1.5 interquartile range. All ChIP-seq and ATAC-seq experiments, n=2 replicates from independent differentiations.

FIGS. 13a-13e. Class I and class II pancreatic enhancers exhibit distinct enhancer architecture. FIG. 13a. Selected FOXA1 and FOXA2 motifs and associated position weight matrices (PWMs) obtained from JASPAR. FIG. 13b. Probability (occurrence per base pair) of FOXA1 (MA0148.1 and MA0148.4) and FOXA2 (MA0047.1 and MA0047.3) motifs relative to ATAC-seq peak summits at class I (solid line) and class II (dashed line) enhancers. ATAC-seq peak summits at class I enhancers are enriched for occurrences compared to summits at class II enhancers (P=8.4×10⁻¹⁵, 1.6×10⁻³, 1.3×10⁻³, and 2.1×10⁻⁵ for MA0148.1, MA0148.4, MA0047.1, and MA0047.3, respectively; Fisher's exact test, 1-sided). FIG. 13c. mRNA expression levels of pancreatic transcription factors (TF) determined by RNA-seq. Data are shown as mean fragments per kilobase per million fragments mapped (FPKM) ±S.E.M. in ES, DE, GT, PP1, and PP2 (n=3 independent differentiations). FIG. 13d. Percentage of class I and class II enhancers overlapping HNF6, PDX1, and SOX9 ChIP-seq peaks (within 100 bp from peak) in PP2. FIG. 13e. Heatmap showing enriched de novo TF binding motifs at HNF6-, PDX1-, and SOX9-bound class I against a background of HNF6-, PDX1-, and SOX9-bound class II enhancers and vice versa. Fisher's exact test, 1-sided, corrected for multiple comparisons.

FIGS. 14a-14d. Characterization of class I and class II enhancers in PDX1-deficient pancreatic progenitors. (a) qPCR analysis of PDX1, FOXA1, and FOXA2 in PP2 cells differentiated from hESCs transduced with a scrambled control (SCRAM) or PDX1 shRNA (shPDX1) lentivirus (n=2 independent differentiations; P=5.90×10⁻⁵, 2.74×10⁻³, and 0.883 for PDX1, FOXA1, and FOXA2, respectively, in SCRAM compared to shPDX1 PP2 cells; student's t-test, 2-sided). Bar graph shows mean±S.E.M. (b) Percentage of PDX1-bound class I and class II enhancers exhibiting a significant loss of FOXA1/2 ChIP-seq signal (≥2-fold decrease, P adj.<0.05) in shPDX1 vs SCRAM PP2 (P<2.2×10⁻¹⁶; Fisher's exact test, 2-sided). (c) Box plot of fold change in FOXA1/2 ChIP-seq signal at PDX1-bound class I and class II enhancers in shPDX1 vs SCRAM PP2 cells. (P=<2.2×10⁻¹⁶; Wilcoxon rank sum test, 2-sided). (d) Box plots of ATAC-seq and H3K27ac ChIP-seq counts at class I and class II pancreatic enhancers in SCRAM or shPDX1 PP2 cells (P=<2.2×10⁻¹⁶, <2.2×10⁻¹⁶, 7.03×10⁻¹³, and <2.2×10⁻¹⁶ for SCRAM versus shPDX1 of ATAC signal at class I enhancers, ATAC signal at class II enhancers, H3K27ac signal at class I enhancers, and H3K27ac signal at class II enhancers, respectively; Wilcoxon rank sum test, 2-sided). All box plots are centered on median, with box encompassing 25th-75th percentile and whiskers extending up to 1.5 interquartile range. All ChIP-seq and ATAC-seq experiments, n=2 replicates from independent differentiations.

FIGS. 15a-15g. Optimization of FOXA binding motifs alters target gene expression and cell fate. FIG. 15a. Feature plots of single cell RNA-seq data showing expression of selected marker genes across cell populations at PP2. UMAPs shown are integrated across control and motif optimized cells. FIG. 15b. Dot plot showing expression levels of NKX6.1 across cell populations in control and motif optimized PP2 cells. FIG. 15c. Dot plot showing expression levels of select marker genes in NKX6.1-expressing cells in control and motif optimized PP2 cells. FIG. 15d. qPCR analysis of NKX6.1 in control and motif optimized GT and PP2 cells (n=3 independent differentiations; P=0.39 and 0.19 for comparisons at GT and PP2, respectively; student's t-test, 2-sided). Line graph shows mean±S.E.M. FIG. 15e. Immunofluorescent staining for PDX1 and NKX6.1 in PP2 control and motif optimized cells. Scale bars, 50 μm. Representative images are shown from n≥2 independent differentiations. FIG. 15f. Schematic illustrating differentiation from the late pancreatic progenitor stage (PP2) to the endocrine progenitor stage (EN). FIG. 15g. qPCR analysis of GCG (top), and flow cytometry quantification (bottom) of insulin⁺ and NKX6.1⁺ cells in control and motif optimized EN cells (qPCR: n=2 independent differentiations, each plotted point represents the average of 3 technical replicates, P=8.9×10⁻³; flow cytometry: n=3 independent differentiations, P=2.2×10⁻³; student's t-test, 2-sided). Bar graphs show mean±S.E.M. Multipotent pancreatic progenitor cells, MPCs; early endocrine progenitor cells, EPCs.

FIGS. 16a-16f. Identification and characterization of hepatic and alveolar enhancers. FIG. 16a. Volcano plot showing identification of hepatic enhancers based on increase in H3K27ac signal from GT to HP (≥2-fold increase, P adj.<0.05 at sites >2.5 kb from TSS; DESeq2). Enriched gene ontology terms of genes linked to hepatic enhancers using GREAT. FIG. 16b. Box plots of H3K27ac ChIP-seq counts at hepatic enhancers. FIG. 16c. Volcano plot showing identification of alveolar enhancers based on increase in H3K27ac signal from AFG to ALV (≥2-fold increase, P adj.<0.05 at sites >2.5 kb from TSS; DESeq2). Enriched gene ontology terms of genes linked to alveolar enhancers using GREAT. FIG. 16d. Box plots of H3K27ac ChIP-seq counts at alveolar enhancers. FIG. 16e and FIG. 16f. Heatmaps showing density of H3K27ac ChIP-seq reads at hepatic (FIG. 16e) and alveolar (FIG. 16f) class I and class II enhancers in GT and HP (FIG. 16e) and AFG and ALV (FIG. 16f). Heatmaps are centered on H3K27ac peaks and span 5 kb. All box plots are centered on median, with box encompassing 25th-75th percentile and whiskers extending up to 1.5 interquartile range. All ChIP-seq experiments, n=2 replicates from independent differentiations.

FIGS. 17a-17g FOXA1/2 binding sites at class I and class II hepatic and alveolar enhancers differ in DNA sequence. FIG. 17a. mRNA expression levels of hepatic transcription factors (TF) determined by RNA-seq. Data are shown as mean fragments per kilobase per million fragments mapped (FPKM) ±S.E.M. in ES, DE, GT (n=3 independent differentiations), and HP (n=1 differentiation). FIG. 17b and FIG. 17c. Percentage of class I and class II hepatic (FIG. 17b) and alveolar (FIG. 17c) enhancers with at least one occurrence of selected FOXA1 and FOXA2 motifs (P=<2.2×10⁻¹⁶, <2.2×10⁻¹⁶, 2.42×10⁻¹², 7.81×10⁻¹¹, <2.2×10⁻¹⁶, and 1.32×10⁻¹⁴ for comparisons of occurrences of MA0148.1, MA0148.3, MA0148.4, MA0047.1, MA0047.2, and MA0047.3, respectively, at hepatic enhancers. P=4.88×10⁻¹³, 2.11×10⁻¹³, 6.17×10⁻⁹, 8.89×10⁻⁹, 2.2×10⁻¹⁶, and 9.75×10⁻¹¹ for comparisons of occurrences of MA0148.1, MA0148.3, MA0148.4, MA0047.1, MA0047.2, and MA0047.3, respectively, at alveolar enhancers. Fisher's exact test, 2-sided). FIG. 17d. Probability (occurrences per base pair) of FOXA1 (MA0148.1, MA0148.3, MA0148.4) and FOXA2 (MA0047.1, MA0047.2, MA0047.3) motifs relative to ATAC-seq peak summits at class I (solid line) and class II (dashed line) hepatic enhancers. ATAC-seq peak summits at class I enhancers are enriched for occurrences compared to summits at class II enhancers (P=4.3×10⁻¹⁴, 3.3×10⁻¹⁹, 2.0×10⁻³, 5.5×10⁻⁴, 6.7×10⁻¹⁷, and 2.1×10⁻⁵ for MA0148.1, MA0148.3, MA0148.4, MA0047.1, MA0047.2, and MA0047.3, respectively; Fisher's exact test, 1-sided). FIG. 17e. Percentage of hepatic class I and class II enhancers containing FOXA TF ATAC-seq footprints in HP (P=1.01×10⁻¹⁰ for comparison of class I and class II enhancers; Fisher's exact test, 2-sided). FIG. 17f. Percentage of hepatic class I and class II enhancers overlapping GATA4 and GATA6 ChIP-seq peaks in GT and HNF4A ChIP-seq peaks (within 100 bp from peak) in HP (P=<2.2×10⁻¹⁶, <2.2×10⁻¹⁶, and 2.51×10⁻⁴ for comparisons of GATA4, GATA6, and HNF4A, respectively; Fisher's exact test). FIG. 17g. Enriched de novo TF binding motifs at HNF4A-bound class I against a background of HNF4A-bound class II enhancers and vice versa. Fisher's exact test, 1-sided, corrected for multiple comparisons. All ChIP-seq experiments, n=2 replicates from independent differentiations.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

The present disclosure provides a method of improving the yield of insulin-producing cells produced from pluripotent stem cells for transplantation into patients with diabetes. More specially, the present disclosure provides a clonal human pluripotent stem cell line that broadly expresses the gene NKX6.1 across cells at the pancreatic progenitor cell stage of in vitro differentiation, which yields fewer alpha cells and more beta cells during directed differentiation to islet cells. Therefore, the present disclosure provides a method of treating diabetes using the beta cells produced from the method disclosed in the present disclosure.

The present disclosure provides that FOXA pioneer transcription factors (TFs) associate with primed enhancers in endodermal organ precursors. In certain embodiments, the present disclosure provides that, using a human stem cell model of pancreas differentiation, it is discovered that only a subset of pancreatic enhancers is FOXA-primed, whereas the majority is unprimed and engages FOXA upon lineage induction. Primed enhancers are enriched for signal-dependent TF motifs and harbor abundant and strong FOXA motifs. Unprimed enhancers harbor fewer, more degenerate FOXA motifs, and FOXA recruitment to unprimed but not primed enhancers requires pancreatic TFs. Strengthening FOXA motifs at an unprimed enhancer near NKX6.1 renders FOXA recruitment pancreatic TF-independent, induces priming, and broadens the NKX6.1 expression domain. Further, the present disclosure provides the FOXA binding during hepatic and lung development. A dual role for FOXA in endodermal organ development is provided herein: first, FOXA facilitates signal-dependent lineage initiation via enhancer priming, and second, FOXA enforces organ cell type-specific gene expression via indirect recruitment by lineage-specific TFs.

Accordingly, the present disclosure provides a method of engineering cells, such as hESCs, for producing more precursor beta cells. In certain embodiments, the present disclosure provides a method of identifying and modifying sites in an enhancer for NKX6.1 which led to an increase in precursors for pancreatic insulin-producing beta cells. The present disclosure further provides a method of treating diabetes, particularly diabetes type I, using the genetically modified hESC cells and the resulting precursor beta cells.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a catalyst,” “a metal,” or “a substrate,” includes, but are not limited to, mixtures or combinations of two or more such catalysts, metals, or substrates, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Unless otherwise specified, temperatures referred to herein are based on atmospheric pressure (i.e. one atmosphere).

FOXA TFs are generally thought to control developmental transitions by mediating chromatin priming owing to FOXA's pioneer TF activity^{1, 11, 38}. It was previously reported that chromatin priming and FOXA1/2 recruitment precede organ lineage induction at pancreas, liver, and lung enhancers¹. Here, it shows that chromatin priming and early FOXA1/2 recruitment are limited to a small subset of organ lineage enhancers, whereas the majority transitions from unprimed to active and engages FOXA1/2 upon lineage induction. It is demonstrated that DNA sequence logic is the primary determinant of whether an enhancer is primed and recruits FOXA1/2 independent of lineage-specific TFs or whether it is unprimed and requires lineage-specific TFs for FOXA1/2 binding. The results presented here provide a molecular framework for understanding gene regulatory principles that underlie lineage induction and cell type diversification during organogenesis. These findings support a model whereby FOXA-mediated priming of a subset of organ-specific enhancers enables the initiation of organ-specific gene expression programs by lineage-inductive cues, whereas secondary recruitment of FOXA by lineage-specific TFs to most organ-specific enhancers helps establish cell type-specific gene expression by safeguarding against broad target gene expression within the organ progenitor domain.

Stronger and more abundant FOXA motifs were observed at primed compared to unprimed enhancers and it was found that FOXA1/2 recruitment to a proportion of unprimed enhancers depends on the pancreatic TF PDX1. Furthermore, it shows that strengthening FOXA motifs at an unprimed enhancer obviates dependency of FOXA1/2 binding on PDX1, resulting in FOXA recruitment and enhancer priming prior to lineage induction. These findings are consistent with prior observations in tumor cell line models, which have suggested that the ability of FOXA TFs to stably bind and remodel chromatin is DNA sequence-dependent^{17, 18, 20}. The results presented herein extend these observations in immortalized cell lines to demonstrate relevance of distinct mechanisms of FOXA recruitment for developmental gene regulation.

The observation that FOXA1/2 bind primed enhancers without cooperative recruitment by pancreatic TFs raises the question of how FOXA TFs engage their target sites at primed enhancers. It is found that a subset of primed enhancers is bound by both FOXA and GATA TFs prior to lineage induction. Given previously demonstrated cooperativity between FOXA and GATA TFs¹⁷, it is possible that GATA TFs help recruit FOXA to a subset of primed enhancers. However, it shows that strengthening FOXA motifs is sufficient to enable FOXA1/2 binding to an enhancer not bound by GATA TFs. Therefore, these data support the conclusion that strong FOXA motifs are sufficient to facilitate FOXA TF engagement and chromatin priming during development, consistent with observations that FOXA1/2 can engage target sites on nucleosomal DNA in vitro^{7, 8, 9}.

These findings provide insight into the gene regulatory mechanisms that underlie endodermal organ lineage induction and cell fate specification. It was observed enrichment of binding motifs for signal-dependent TFs and binding of the retinoic acid receptor subunit RXR at primed pancreatic enhancers. These findings suggest that organ lineage-inductive cues are read by primed enhancers to initiate expression of lineage-determining TFs. In support of this, primed enhancers are found near PDX1, HNF1B, and MEIS1, which are among the first TFs expressed upon pancreas induction. By contrast, unprimed enhancers are enriched for binding motifs of organ-specific TFs, which recruit FOXA1/2 secondarily. Given that FOXA TFs are broadly expressed across endodermal organ lineages, indirect FOXA recruitment by organ-specific TFs provides a safeguard against lineage-aberrant enhancer activation and gene expression. This agrees with studies in Drosophila and Ciona, which suggest that suboptimization of TF-binding motifs could be a general principle by which to confer cell specificity to enhancers^{26, 39}.

Replacing low-affinity FOXA-binding sites at an unprimed enhancer for NKX6.1 with higher affinity sites broadened the domain of NKX6.1 expression among pancreatic progenitors. As shown, NKX6.1 was not prematurely expressed, demonstrating that motif optimization does not eliminate the dependency of target gene expression on lineage-specific cues. This suggests that early FOXA recruitment through high affinity-binding sites lowers the threshold for target gene expression, which could reflect an increased sensitivity of the enhancer to activation by lineage-specific TFs. Thus, higher thresholds to target gene expression conferred by unprimed enhancers restricts target gene expression to specific cell populations, as enhancer activation only occurs when a specific complement of lineage-specific TFs is present in sufficient concentrations. Gene regulation by unprimed enhancers provides a mechanism for specifying different cell types early in organ development. Small differences in TF expression among early organ progenitors would be sufficient to activate different repertoires of unprimed enhancers, thereby creating divergent gene expression patterns and cell populations. Consistently, it has been shown that PDX1^{high} and PDX1^{low} cells in the early pancreatic epithelium acquire different cell identities⁴⁰.

It demonstrates that conversion of a single enhancer near NKX6.1 from an unprimed to a primed state is sufficient to alter cell fate due to broadened expression of NKX6.1 within the progenitor cell domain. These findings show that in a developmental context, differences in FOXA-binding affinity at enhancers can affect cell fate allocation. It is therefore possible that polymorphisms at FOXA-binding sites determine interindividual differences in endodermal organ cell type composition. Consistently, islet cell type composition is known to vary greatly in humans' and the NKX6.1 enhancer contains twelve known polymorphisms predicted to alter the strength and spacing of FOXA motifs. While the importance of polymorphisms for organ cell type composition remains to be demonstrated, these findings support that FOXA TF motif strength at developmental enhancers provides a tunable threshold for target gene expression.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Methods & Materials

### Cell Lines and Animal Model

### Human Cell Culture Experiments

hESC research was approved by the University of California, San Diego (UCSD), Institutional Review Board and Embryonic Stem Cell Research Oversight Committee (protocol 090165ZX). Human iPSC research was approved by the Boston University Institutional Review Board (protocol H-33122).

### Maintenance of HEK293T Cells

HEK293T cells (female) were cultured in a humidified incubator at 37° C. with 5% CO₂ using Dulbecco's Modified Eagle Medium (Cat #45000-312; 4.5 g/L glucose, [+]1-glutamine, [−] sodium pyruvate) supplemented with 10% fetal bovine serum (FBS) and 1% Penicillin-Streptomycin (Thermo Fisher Scientific, Cat #15140122).

Maintenance and Differentiation of CyT49 hESCs

CyT49 hESCs (male) were maintained and differentiated as described^{1, 42, 43}. Propagation of CyT49 hESCs was carried out by passing cells every 3 to 4 days using Accutase™ (eBioscience) for enzymatic cell dissociation, and with 10% (v/v) human AB serum (Valley Biomedical) included in the hESC media the day of passage. hESCs were seeded into tissue culture flasks at a density of 50,000 cells/cm². hESC media was comprised of DMEM/F12 (VWR) supplemented with 10% (vol/vol) KnockOut™ Serum Replacement XenoFree (Life Technologies), 0.1 mM MEM non-essential amino acids (Life Technologies), 1× GlutaMAX™ I (Life Technologies), 1% (vol/vol) penicillin/streptomycin (Life Technologies), 0.1 mM 2-mercaptoethanol (Life Technologies), 10 ng/mL Activin A (R&D Systems), and 10 ng/mL Heregulin-31 (PeproTech).

Pancreatic differentiation was performed as previously described^{1, 42, 43}. Briefly, a suspension-based culture format was used to differentiate cells in aggregate form. Undifferentiated aggregates of hESCs were formed by re-suspending dissociated cells in hESC maintenance medium at a concentration of 1×10⁶ cells/mL and plating 5.5 mL per well of the cell suspension in 6-well ultra-low attachment plates (Costar). The cells were cultured overnight on an orbital rotator (Innova2000, New Brunswick Scientific) at 95 rpm (0.2×g). After 24 h the undifferentiated aggregates were washed once with RPMI medium and supplied with 5.5 mL of day 0 differentiation medium. Thereafter, cells were supplied with the fresh medium for the appropriate day of differentiation (see below). Cells were continually rotated at 95 rpm (0.2×g), or 105 rpm (0.2×g) on days 4 through 8, and no media change was performed on day 10. Both RPMI (Mediatech) and DMEM High Glucose (HyClone) medium were supplemented with 1× GlutaMAX™ and 1% penicillin/streptomycin. Human activin A, mouse Wnt3a, human KGF, human noggin, and human EGF were purchased from R&D systems. Other added components included FBS (HyClone), B-27® supplement (Life Technologies), Insulin-Transferrin-Selenium (ITS; Life Technologies), TGFβ R1 kinase inhibitor IV (EMD Bioscience), KAAD-Cyclopamine (KC; Toronto Research Chemicals), and the retinoic receptor agonist TTNPB (RA; Sigma Aldrich). Day-specific differentiation media formulations were as follows:
Days 0 and 1: RPMI+0.2% (v/v) FBS, 100 ng/mL Activin, 50 ng/mL mouse Wnt3a, 1:5000 ITS. Days 1 and 2: RPMI+0.2% (v/v) FBS, 100 ng/mL Activin, 1:5000 ITS
Days 2 and 3: RPMI+0.2% (v/v) FBS, 2.5 mM TGFβ R1 kinase inhibitor IV, 25 ng/mL KGF, 1:1000 ITS
Days 3-5: RPMI+0.2% (v/v) FBS, 25 ng/mL KGF, 1:1000 ITS
Days 5-8: DMEM+0.5× B-27® Supplement, 3 nM TTNPB, 0.25 mM KAAD-Cyclopamine, 50 ng/mL Noggin
Days 8-10: DMEM/B-27, 50 ng/mL KGF, 50 ng/mL EGF
Cells at D0 correspond to the embryonic stem cell (ES) stage, cells at D2 correspond to the definitive endoderm (DE) stage, cells at D5 correspond to the gut tube (GT) stage, cells at D7 correspond to the early pancreatic progenitor (PP1) stage, and cells at D10 correspond to the late pancreatic progenitor (PP2) stage.

Hepatic differentiation was performed as previously described¹. Briefly, cells were treated identically as in pancreatic differentiation until the GT stage at D5. At this point cells were treated with 50 ng/mL BMP4 (Millipore) and 10 ng/mL FGF2 (Millipore) in RPMI media (Mediatech) supplemented with 0.2% (vol/vol) FBS (HyClone) for 3 days with daily media changes. Cells at D8 correspond to the hepatic progenitor (HP) cell stage. A full list of reagents and catalog numbers is provided in Table 1.

TABLE 1
Reagents used for maintenance and differentiation of Cyt49 hESCs

| Reagent | Manufacturer | Catalog Number |
|---|---|---|
| 2-Mercaptoethanol | Thermo Fisher Scientific | Cat# 21985023 |
| Accutase | Thermo Fisher Scientific | Cat# 00-4555-56 |
| Activin A | R&D Systems | Cat# 338-AC/CF |
| B-27 supplement | Thermo Fisher Scientific | Cat# 17504044 |
| DMEM High Glucose | VWR | Cat# 16750-082 |
| DMEM/F12 [−] | VWR | Cat# 15-090-CV |
| L-glutamine | | |
| Fetal Bovine Serum | Thermo Fisher Scientific | Cat# MT35011CV |
| GlutaMAX | Thermo Fisher Scientific | Cat# 35050061 |
| Human AB Serum | Valley Biomedical | Cat# HP1022 |
| Insulin-Transferrin- | Thermo Fisher Scientific | Cat# 41400045 |
| Selenium (ITS) | | |
| KAAD-Cyclopamine | Toronto Research | Cat# K171000 |
| | Chemicals | |
| KnockOut SR XenoFree | Thermo Fisher Scientific | Cat# A1099202 |
| Matrigel ® | Corning | Cat# 356231 |
| Non-Essential Amino | Thermo Fisher Scientific | Cat# 11140050 |
| Acids | | |
| Penicillin-Streptomycin | Thermo Fisher Scientific | Cat# 15140122 |
| Recombinant EGF | R&D Systems | Cat# 236-EG |
| Recombinant Heregulinβ-1 | Peprotech | Cat# 100-03 |
| Recombinant human | R&D Systems | Cat#314-BP |
| BMP4 | | |
| Recombinant KGF/FGF7 | R&D Systems | Cat# 251-KG |
| Recombinant Mouse | R&D Systems | Cat# 1324-WN/ |
| Wnt3A | | CF |
| Recombinant Noggin | R&D Systems | Cat# 3344NG |
| RPMI 1640 [−] L-glutamine | VWR | Cat# 15-040-CV |
| TGF-β RI Kinase | Calbiochem | Cat# 616454 |
| Inhibitor IV | | |
| TTNPB | Enzo Life Sciences | Cat# BML-GR105 |

Maintenance and Differentiation of H1 hESCs

H1 hESCs (male) were maintained and differentiated as described with some modifications A^{44, 45}. In brief, hESCs were cultured in mTeSR1 media (Stem Cell Technologies) supplemented with 1% Penicillin-Streptomycin (Thermo Fisher Scientific, Cat #15140122) and propagated by passaging cells onto 6-well plates coated with Matrigel (Corning) every 3 to 4 days using Accutase (eBioscience) for enzymatic cell dissociation.

For differentiation, cells were dissociated using Accutase for 10 min, then reaggregated in mTESR supplemented with Y-27632 (Stem Cell Technologies) by plating the cells at a concentration of ˜5.5×10⁶ cells/well in a low attachment 6-well plate on an orbital shaker (100 rpm, 0.2×g) in a 37° C. incubator. The following day, undifferentiated cells were washed in base media (see below) and then differentiated using a multi-step protocol with stage-specific media and daily media changes.

All stage-specific base media were comprised of MCDB 131 medium (Thermo Fisher Scientific) supplemented with NaHCO₃, GlutaMAX, D-Glucose, and BSA using the following concentrations:
Stage 1/2 base medium: MCDB 131 medium, 1.5 g/L NaHCO₃, 1× GlutaMAX, 10 mM D-Glucose, 0.5% BSA
Stage 3/4 base medium: MCDB 131 medium, 2.5 g/L NaHCO₃, 1× GlutaMAX, 10 mM D-glucose, 2% BSA
Stage 5 medium: MCDB 131 medium, 1.5 g/L NaHCO₃, 1× GlutaMAX, 20 mM D-glucose, 2% BSA

Media compositions for each stage were as follows:
Stage 1 (days 0-2): base medium, 100 ng/mL Activin A, 25 ng/mL Wnt3a (day 0). Day 1-2: base medium, 100 ng/mL Activin A
Stage 2 (days 3-5): base medium, 0.25 mM I-Ascorbic Acid (Vitamin C), 50 ng/mL FGF7
Stage 3 (days 6-7): base medium, 0.25 mM I-Ascorbic Acid, 50 ng/mL FGF7, 0.25 μM SANT-1, 1 μM Retinoic Acid, 100 nM LDN193189, 1:200 ITS-X, 200 nM TPB
Stage 4 (days 8-10): base medium, 0.25 mM I-Ascorbic Acid, 2 ng/mL FGF7, 0.25 μM SANT-1, 0.1 μM Retinoic Acid, 200 nM LDN193189, 1:200 ITS-X, 100 nM TPB
Stage 5 (days 11-13): base medium, 0.25 μM SANT-1, 0.05 μM RA, 100 nM LDN-193189, 1 μM T3, 10 μM ALK5i II, 10 μM ZnSO4, 10 μg/mL heparin, 1:200 ITS-X
Cells at DO, D3, D6, D8, D11, and D14 correspond to the ES DE, GT, PP1, PP2, and EN stages, respectively. At D8 of differentiation, speed of the orbital shaker was increased to 110 rpm (0.3×g). A full list of reagents and catalog numbers is provided in Table 2.

TABLE 2
Reagents used for maintenance and differentiation of H1 hESCs

| Reagent | Manufacturer | Catalog Number |
|---|---|---|
| 3,3′,5-Triiodo-L- | Sigma-Aldrich | Cat# T6397-100 MG |
| thyronine | | |
| sodium salt (T3) | | |
| Accutase | Thermo Fisher Scientific | Cat# 00-4555-56 |
| Activin A | R&D Systems | Cat# 338-AC/CF |
| Alk5 Inhibitor II | Cayman Chemicals | Cat# 14794 |
| Bovine Albumin | Life Technologies | Cat# 15260037 |
| Fraction V | | |
| D-(+)-Glucose Solution, | Sigma-Aldrich | Cat# G8769 |
| 45% | | |
| GlutaMAX | Thermo Fisher Scientific | Cat# 35050061 |
| Heparin | Sigma-Aldrich | Cat# H3149-250KU |
| Insulin-Transferrin- | Thermo Fisher Scientific | Cat# 51500-056 |
| Selenium-Ethanolamine | | |
| (ITS-X) | | |
| L-Ascorbic Acid | Sigma-Aldrich | Cat# A4544 |
| LDN-193189 | Stemgent | Cat# 04-0074 |
| Matrigel ® | Corning | Cat# 356231 |
| MCDB 131 | Thermo Fisher Scientific | Cat# 10372-019 |
| mTeSR1 Complete | Stem Cell Technologies | Cat# 85850 |
| Kit-GMP | | |
| Penicillin-Streptomycin | Thermo Fisher Scientific | Cat# 15140122 |
| Recombinant KGF/FGF7 | R&D Systems | Cat# 251-KG |
| Recombinant Mouse | R&D Systems | Cat# 1324-WN/CF |
| Wnt3A | | |
| Retinoic Acid | Sigma-Aldrich | Cat# R2625 |
| ROCK Inhibitor Y-27632 | Stem Cell Technologies | Cat# 72305 |
| SANT-1 | Sigma-Aldrich | Cat# S4572 |
| Sodium Bicarbonate | Sigma-Aldrich | Cat# NC0564699 |
| TPB | Calbiochem | Cat# 565740 |
| Zinc sulfate heptahydrate | Sigma-Aldrich | Cat# Z0251 |
| (ZnSO4) | | |

Maintenance and Differentiation of iPSCs

SPC2 iPSCs (male; clone SPC2-ST-1B⁴⁶) were maintained in feeder-free culture conditions in 6-well tissue culture dishes (Corning) coated with growth factor-reduced Matrigel (Corning, Cat #356231), in mTeSR1 medium (Stem Cell Technologies, Cat #85850) and passaged using gentle cell dissociation reagent (GCDR; Stem Cell Technologies, Cat #07174). Details of iPSC derivation, characterization, and differentiation into anterior foregut endoderm and alveolar epithelial type 2 cells (iAT2s; also known as iAEC2s) have been previously published^{31, 46, 47} and are available for free download. Briefly, the SPC2-ST-B2 iPSC clone, engineered to carry a tdTomato reporter knocked into one allele of the endogenous SFTPC locus⁴⁶, underwent directed differentiation to generate iAT2s in 3D Matrigel cultures as follows. Cells were first differentiated into definitive endoderm using the STEMdiff Definitive Endoderm Kit (Stem Cell Technologies, Cat #05110) for 72 h and subsequently dissociated with GCDR and passaged as small clumps into growth factor-reduced Matrigel-coated (Corning, Cat #356231) 6-well culture plates (Corning) in “DS/SB” foregut endoderm anteriorization media, consisting of complete serum-free differentiation medium (cSFDM) base as previously described³¹, supplemented with 10 μm SB431542 (“SB”; Tocris, Cat #1614) and 2 μm Dorsomorphin (“DS”; Stemgent, Cat #04-0024), to pattern cells towards anterior foregut endoderm (AFE; day 6 of differentiation). For the first 24 h after passaging, media was supplemented with 10 μM Y-27632 (Stem Cell Technologies, Cat #72305). After anteriorization in DS/SB media for 72 h, beginning on day 6 of differentiation cells were cultured in “CBRa” lung progenitor-induction medium for 9 additional days. “CBRa” medium consists of cSFDM base supplemented with 3 μM CHIR99021 (Tocris, Cat #4423), 10 ng/mL recombinant human BMP4 (rhBMP4; R&D Systems, Cat #314-BP), and 100 nM retinoic acid (RA; Sigma, Cat #R2625), as described³¹. On differentiation day 15, NKX2-1⁺ lung progenitors were isolated based on CD47^{hi}/CD26^{neg} gating⁴⁸ using a high-speed cell sorter (MoFlo Legacy or MoFlo Astrios EQ). Purified day 15 lung progenitors were resuspended in undiluted growth factor-reduced 3D Matrigel (Corning, Cat #356231) at a concentration of 400 cells/μL and distal/alveolar differentiation was performed in “CK+DCI” medium, consisting of cSFDM base supplemented with 3 μm CHIR99021 (Tocris, Cat #4423), 10 ng/mL rhKGF (R&D Systems, Cat #251-KG), and 50 nM dexamethasone (Sigma, Cat #D4902), 0.1 mM 8-Bromoadenosine 30, 50-cyclic monophosphate sodium salt (Sigma, Cat #B7880) and 0.1 mM 3-Isobutyl-1-methylxanthine (IBMX; Sigma, Cat #15879) (DCI) with a brief period of CHIR99021 withdrawal between days 34-39 to achieve iAT2 maturation. To establish pure cultures of iAT2s, cells were sorted by flow cytometry on day 45 to purify SFTPC^{tdTomato+} cells. iAT2s were maintained as self-renewing monolayered epithelial spheres (“alveolospheres”) through serial passaging every 10-14 days and replating in undiluted growth factor-reduced 3D Matrigel (Corning, Cat #356231) droplets at a density of 400 cells/μl in CK+DCI medium, as described⁴⁷. iAT2 culture quality and purity was monitored at each passage by flow cytometry, with 95.2±4.2% (mean±S.D.) of cells expressing SFTPC^{tdTomato} over time, as we have previously detailed^{31, 45}.

Cells at day 6 correspond to the AFG stage and day 261 iAT2s were used for the alveolar stage.

Generation of FOXA1^{-/-}, FOXA2^{-/-}, and FOXA1/2^{-/-} H1 hESC Lines

To generate homozygous FOXA1, FOXA2, and FOXA1/2 deletion hESC lines, sgRNAs targeting coding exons within each gene were cloned into Px333-GFP, a modified version of Px333³⁹, which was a gift from Andrea Ventura (Addgene, #64073). The plasmid was transfected into H1 hESCs with XtremeGene 9 (Roche, Cat #6365787001), and 24 h later 8000 GFP⁺ cells were sorted into a well of six-well plate. Individual colonies that emerged within 5-7 days were subsequently transferred manually into 48-well plates for expansion, genomic DNA extraction, PCR genotyping, and Sanger sequencing. For control clones, the Px333-GFP plasmid was transfected into H1 hESCs, and cells were subjected to the same workflow as H1 hESCs transfected with sgRNAs.

sgRNA oligo used to generate FOXA1^{-/-} hESCs:

(SEQ ID NO: 1)
CGCCATGAACAGCATGACTG

sgRNA oligo used to generate FOXA2^{-/-} hESCs:

(SEQ ID NO: 2)
CATGAACATGTCGTCGTACG

sgRNA oligos used to generate FOXA1/2^{-/-} frameshift hESCs:

(SEQ ID NO: 1)
FOXA1: CGCCATGAACAGCATGACTG
(SEQ ID NO: 2)
FOXA2: CATGAACATGTCGTCGTACG

sgRNA oligos used to generate FOXA1/2^{-/-} exon deletion hESCs:

(SEQ ID NO: 3)
FOXA1 upstream: GCGACTGGAACAGCTACTAC
(SEQ ID NO: 4)
FOXA1 downstream: GCACTGCAATACTCGCCTTA
(SEQ ID NO: 5)
FOXA2 upstream: TCCGACTGGAGCAGCTACTA
(SEQ ID NO: 6)
FOXA2 downstream: CGGCTACGGTTCCCCCATGC

Generation of NKX6.1 Enhancer Motif Optimized H1 hESC Line

To generate base substitutions in the NKX6.1 enhancer, a sgRNA targeting the enhancer was cloned into the Px458 plasmid⁵⁰, which was a gift from Feng Zhang (Addgene, #48138). The plasmid and an asymmetric single-stranded oligodeoxynucleotide donor template (ssODN) were transfected into H1 hESCs with XtremeGene 9 (Roche, Cat #6365787001), and cells were treated with 1 μM SCR7 DNA ligase IV inhibitor (Stem Cell Technologies, Cat #74102) to promote homology-directed repair. Twenty-four hours later 8000 GFP⁺ cells were sorted into a well of six-well plate. Individual colonies that emerged within 5-7 days were subsequently transferred manually into 48-well plates for expansion, genomic DNA extraction, PCR genotyping, and Sanger sequencing.

sgRNA Oligo Used to Target NKX6.1 Enhancer:

(SEQ ID NO: 7)
AAAACAATCTGAGGAGAACA

ssODN Sequence:

(SEQ ID NO: 8)
TGCCTATGATTTATGTATTTGTTTAGTCAATAGTCTAATGTAAATGATGT
AATTAATTATAGATGGTGGTGTCAGGTCATTTGTGTAAACAATCTGAGGT
AAACAAGGGCTCTGTTTACTTCATGACAGATGCAGGGGGGGGGGGGCTGA
GTTGAGGGAATTCCAGGGGAACTTTTTCACGTGTGAATGGCGGCTGGGA

Transduction of CyT49 hESCs with SCRAM and shPDX1

To generate shRNA expression vectors, shRNA guide sequences were placed under the control of the human U6 pol III promoter in the pLL3.7 backbones⁵¹, which was a gift from Luk Parijs (Addgene, plasmid #11795). Short hairpin sequences are provided in Table 3.

TABLE 3
Short hairpin sequences used for PDX1 knockdown

| | Sense | Antisense |
|---|---|---|
| SCRAM | 5′- | 5′- |
| | TGAACAAGATGAAGAGCACCTTC | TCGAGAAAAAAGAACAAGATGAAGAGCAC |
| | AAGAGAGGTGCTCTTCATCTTGT | CTCTCTTGAAGGTGCTCTTCATCTTGTCA- |
| | TCTTTTTTC-3′ (SEQ ID NO: 9) | 3′ (SEQ ID NO: 10) |
| shPDX1 | 5′- | 5′- |
| | TGGAGTTCCTATTCAACAAGTTC | TCGAGAAAAAAGGAGTTCCTATTCAACAAG |
| | AAGAGACTTGTTGAATAGGAACT | TCTCTTGAACTTGTTGAATAGGAACTCA-3′ |
| | CCTTTTTTC-3′ (SEQ ID NO: 11) | (SEQ ID NO: 12) |

High-titer lentiviral supernatants were generated by co-transfection of the shRNA expression vector and the lentiviral packaging construct into HEK293T cells as described⁴². Briefly, shRNA expression vectors were co-transfected with the pCMV-R8.74 and pMD2.G expression plasmids (Addgene #22036 and #12259, respectively, gifts from Didier Trono) into HEK293T cells using a 1 mg/mL PEI solution (Polysciences, Cat #23966-1). Lentiviral supernatants were collected at 48 h and 72 h after transfection. Lentiviruses were concentrated by ultracentrifugation for 120 min at 68,567×g using a Beckman SW28 ultracentrifuge rotor at 4° C.

CyT49 hESCs were plated onto a six-well plate at a density of 1 million cells per well. The following morning, concentrated lentivirus was added at 5 L/mL media, as well as 8 μg/mL polybrene (Fisher Scientific, Cat #TR1003G). After 30 min of incubation, the 6-well plate was spun in a centrifuge (Sorvall Legend RT) for 1 h at 30° C. at 950×g. 6 h later, viral media was replaced with fresh base culture media. After 72 h, cells were sorted for GFP expression and re-cultured.

### Immunofluorescence Analysis

Cell aggregates derived from hESCs were allowed to settle in microcentrifuge tubes and washed twice with PBS before fixation with 4% paraformaldehyde (PFA) for 30 min at room temperature. Fixed samples were washed twice with PBS and incubated overnight at 4° C. in 30% (w/v) sucrose in PBS. Samples were then loaded into disposable embedding molds (VWR), covered in Tissue-Tek® O.C.T. Sakura® Finetek compound (VWR) and flash frozen on dry ice to prepare frozen blocks. The blocks were sectioned at 10 μm and sections were placed on Superfrost Plus® (Thermo Fisher) microscope slides and washed with PBS for 10 min. Slide-mounted cell sections were permeabilized and blocked with blocking buffer, consisting of 0.15% (v/v) Triton X-100 (Sigma, Cat #T8787) and 1% (v/v) normal donkey serum (Jackson Immuno Research Laboratories, Cat #017-000-121) in PBS, for 1 h at room temperature. Slides were then incubated overnight at 4° C. with primary antibody solutions. The following day slides were washed five times with PBS and incubated for 1 h at room temperature with secondary antibody solutions. Cells were washed five times with PBS before coverslips were applied.

All antibodies were diluted in blocking buffer at the ratios indicated below. Primary antibodies used were mouse anti-FOXA1 (1:100 or 1:1000 dilution, Abcam ab55178); goat anti-FOXA2 (1:300 dilution, R&D systems AF2400); goat anti-SOX17 (1:300 dilution, R&D systems AF1924); goat anti-HNF4A (1:1000 dilution, Santa Cruz Biotechnology SC-6556); rabbit anti-PDX1 (1:500 dilution, Abcam ab47267); and mouse anti-NKX6.1 (1:300 dilution, Developmental Studies Hybridoma Bank F64A6B4). Secondary antibodies against mouse, rabbit, and goat were Alexa488- and Cy3-conjugated donkey antibodies (Jackson Immuno Research Laboratories, Cat #715-165-150, 711-485-152, and 705-545-003, respectively), and were used at dilutions of 1:500 (anti-rabbit Alexa488) or 1:1000 (all other secondary antibodies). Cell nuclei were stained with Hoechst 33342 (1:3000, Invitrogen, Cat #H3570). Representative images were obtained with a Zeiss Axio-Observer-Z1 microscope equipped with a Zeiss ApoTome and AxioCam digital camera. Figures were prepared in Adobe Creative Suite 5.

### Flow Cytometry Analysis

Cell aggregates derived from hESCs were allowed to settle in microcentrifuge tubes and washed with PBS. Cell aggregates were incubated with Accutase® at 37° C. until a single-cell suspension was obtained. Cells were washed with 1 mL ice-cold flow buffer comprised of 0.2% BSA in PBS and centrifuged at 200×g for 5 min. BD Cytofix/Cytoperm™ Plus Fixation/Permeabilization Solution Kit was used to fix and stain cells for flow cytometry according to the manufacturer's instructions. Briefly, cell pellets were resuspended in ice-cold BD Fixation/Permeabilization solution (300 μL per microcentrifuge tube). Cells were incubated for 20 min at 4° C. Cells were washed twice with 1 mL ice-cold 1×BD Perm/Wash™ Buffer and centrifuged at 10° C. and 200×g for 5 min. Cells were resuspended in 50 μL ice-cold 1×BD Perm/Wash™ Buffer containing diluted antibodies, for each staining performed. Cells were incubated at 4° C. in the dark for 1-3 h. Cells were washed with 1.25 mL ice-cold 1×BD Wash Buffer and centrifuged at 200×g for 5 min. Cell pellets were resuspended in 300 μL ice-cold flow buffer and analyzed in a FACSCanto™ II (BD Biosciences). Antibodies used were PE-conjugated anti-SOX17 antibody (1:20 dilution, BD Biosciences AF1924); mouse anti-HNF1B antibody (1:100 dilution, Santa Cruz Biotechnology sc-130407); PE-conjugated anti-mouse IgG (1:50 dilution, BD Biosciences 555749); PE-conjugated anti-PDX1 (1:10 dilution, BD Biosciences 562161); AlexaFluor® 647-conjugated anti-NKX6.1 (1:5 dilution, BD Biosciences 563338); and PE-conjugated anti-Insulin (1:50 dilution, Cell Signaling 8508). Data were processed using FlowJo software v10.

### Chromatin Immunoprecipitation Sequencing (ChIP-Seq)

ChIP-seq was performed using the ChIP-IT High-Sensitivity kit (Active Motif) according to the manufacturer's instructions. Briefly, for each cell stage and condition analyzed, 5-10×10⁶ cells were harvested and fixed for 15 min in an 11.1% formaldehyde solution. Cells were lysed and homogenized using a Dounce homogenizer and the lysate was sonicated in a Bioruptor® Plus (Diagenode), on high for 3×5 min (30 s on, 30 s off). Between 10 and 30 μg of the resulting sheared chromatin was used for each immunoprecipitation. Equal quantities of sheared chromatin from each sample were used for immunoprecipitations carried out at the same time. Four micrograms of antibody were used for each ChIP-seq assay. Chromatin was incubated with primary antibodies overnight at 4° C. on a rotator followed by incubation with Protein G agarose beads for 3 h at 4° C. on a rotator. Antibodies used were rabbit anti-H3K27ac (Active Motif 39133); rabbit anti-H3K4me1 (Abcam ab8895); goat anti-FOXA1 (Abcam Ab5089); goat-anti-FOXA2 (Santa Cruz SC-6554); goat anti-GATA4 (Santa Cruz SC-1237); mouse anti-GATA6 (Santa Cruz SC-9055); and mouse anti-HNF4A (Novus PP-H1415). Reversal of crosslinks and DNA purification were performed according to the ChIP-IT High-Sensitivity instructions, with the modification of incubation at 65° C. for 2-3 h, rather than at 80° C. for 2 h. Sequencing libraries were constructed using KAPA DNA Library Preparation Kits for Illumina® (Kapa Biosystems) and library sequencing was performed on either a HiSeq 4000 System (Illumina®) or NovaSeq 6000 System (Illumina®) with single-end reads of either 50 or 75 base pairs (bp). Sequencing was performed by the UCSD Institute for Genomic Medicine (IGM) core research facility. For ChIP-seq experiments at the DE, AFG, and ALV stages in iAEC2 cells, two technical replicates from a single differentiation were generated. For all other ChIP-seq experiments, replicates from two independent hESC differentiations were generated.

### ChIP-qPCR

For ChIP-qPCR, immunoprecipitation, reversal of crosslinks, and DNA purification were performed as for ChIP-seq. Antibodies used were rabbit anti-H3K27ac (Active Motif 39133); rabbit anti-H3K4me1 (Abcam ab8895); goat anti-FOXA1 (Abcam Ab5089); and goat anti-FOXA2 (R&D AF2400). After DNA purification, each sample and a 1% dilution of input DNA used for immunoprecipitation were amplified using 2 independent primers targeting either the histones flanking the NKX6.1 enhancer (for measurements of H3K4me1 and H3K27ac) or the FOXA-binding site (for measurements of FOXA1 and FOXA2), as well as a negative control region. qPCR reactions were performed in technical triplicates using a CFX96™ Real-Time PCR Detection System and the iQ™ SYBR® Green Supermix (Bio-Rad, Cat #1708880). A complete list of primer sequences is provided in Table 4.

TABLE 4
Primer sequences used for ChIP-qPCR

| | |
|---|---|
| Flanking histones #1 forward | AAA GGG GAA GGG AGC ACA TG (SEQ ID NO: 13) |
| Flanking histones #1 reverse | TCC TGC ACA CTG TCT ACT GAT (SEQ ID NO: 14) |
| Flanking histones #2 forward | GCT GGG ATC TCA ATT TGC ATG T (SEQ ID NO: 15) |
| Flanking histones #2 reverse | AGG GAG CAG CTG GTT CTT TA (SEQ ID NO: 16) |
| FOXA binding site #1 forward | AAT TAT AGA TGG TGG TGT CAG GT (SEQ ID NO: 17) |
| FOXA binding site #1 reverse | AAA TTG AGA TCC CAG CCG CC (SEQ ID NO: 18) |
| FOXA binding site #2 forward | GAT GGC ACA CTG ACA GAA GAT (SEQ ID NO: 19) |
| FOXA binding site #2 reverse | GCA GAG TCA ATA GGT GCA GAC (SEQ ID NO: 20) |
| Negative control region forward | TCA AAG GCT CAT CTT TGC AG (SEQ ID NO: 21) |
| Negative control region reverse | AAA GCT GGA CTG GTG AAT GC (SEQ ID NO: 22) |

### ChIP-Seq Data Analysis

ChIP-seq reads were mapped to the human genome consensus build (hg19/GRCh37) and visualized using the UCSC Genome Browser⁵². Burrows-Wheeler Aligner (BWA)⁵³ version 0.7.13 was used to map data to the genome. Unmapped and low-quality (q<15) reads were discarded. SAMtools⁵⁴ version 1.5 was used to remove duplicate sequences and HOMER⁵⁵ version 4.10.4 was used to call peaks using the findPeaks command with default parameters. The command “-style factor” was used for TFs and the command “-style histone” was used for histone modifications. Stage- and condition-matched input DNA controls were used as background when calling peaks. The BEDtools⁵⁶ version 2.26.0 suite of programs was used to perform genomic algebra operations. Tag directories were created for each replicate using HOMER. Directories from each replicate were then combined, and peaks were called from the combined replicates using HOMER. These peaks were then intersected with pancreatic enhancers, hepatic enhancers, or alveolar enhancers, respectively. Pearson correlations for the intersecting peaks were calculated between each pair of replicates using the command multiBamSummary from the deepTools2 package⁵⁷ version 3.1.3. Correlations are provided in Table 5.

TABLE 5
Pearson correlation coefficients of ChIP-seq replicates

| | H3K2 | H3K4 | | | | | | | | HN |
|---|---|---|---|---|---|---|---|---|---|---|
| | 7ac | me1 | FOXA1 | FOXA2 | PDX1 | HNF6 | SOX9 | GATA4 | GATA6 | F4A |
| CyT49 ES | 0.94 | N/A | N/A | N/A | N/A | N/A | N/A | N/A | N/A | N/A |
| CyT49 DE | 0.91 | N/A | N/A | N/A | N/A | N/A | N/A | N/A | N/A | N/A |
| CyT49 GT | 0.88 | 0.98 | 0.90 | 0.90 | N/A | N/A | N/A | 0.88 | 0.87 | N/A |
| CyT49 | 0.83 | N/A | N/A | N/A | N/A | N/A | N/A | N/A | N/A | N/A |
| PP1 | | | | | | | | | | |
| CyT49 PP2 | 0.77 | 0.99 | 0.89 | 0.84 | 0.64 | 0.93 | 0.86 | N/A | N/A | N/A |
| CyT49 HP | 0.94 | N/A | 0.84 | 0.70 | N/A | N/A | N/A | N/A | N/A | 0.88 |
| CyT49 | 0.71 | N/A | 0.90 | 0.68 | N/A | N/A | N/A | N/A | N/A | N/A |
| PP2 | | | | | | | | | | |
| SCRAM | | | | | | | | | | |
| CyT49 | 0.86 | N/A | 0.86 | 0.62 | N/A | N/A | N/A | N/A | N/A | N/A |
| PP2 | | | | | | | | | | |
| shPDX1 | | | | | | | | | | |
| H1 GT | 0.98 | 0.86 | N/A | N/A | N/A | N/A | N/A | N/A | N/A | N/A |
| H1 PP2 | 0.97 | 0.88 | N/A | N/A | N/A | N/A | N/A | N/A | N/A | N/A |
| H1 | 0.98 | 0.87 | N/A | N/A | N/A | N/A | N/A | N/A | N/A | N/A |
| FOXA1/2 | | | | | | | | | | |
| exon | | | | | | | | | | |
| deletion | | | | | | | | | | |
| GT | | | | | | | | | | |
| H1 | 0.99 | 0.88 | N/A | N/A | N/A | N/A | N/A | N/A | N/A | N/A |
| FOXA1/2 | | | | | | | | | | |
| exon | | | | | | | | | | |
| deletion | | | | | | | | | | |
| PP2 | | | | | | | | | | |
| iAEC2 DE | 1.00 | N/A | N/A | N/A | N/A | N/A | N/A | N/A | N/A | N/A |
| iAEC2 | 1.00 | N/A | 0.99 | N/A | N/A | N/A | N/A | N/A | N/A | N/A |
| AFG | | | | | | | | | | |
| iAEC2 | 1.00 | N/A | 0.95 | N/A | N/A | N/A | N/A | N/A | N/A | N/A |
| ALV | | | | | | | | | | |

RNA Isolation and Sequencing (RNA-Seq) and qRT-PCR

RNA was isolated from cell samples using the RNeasy® Micro Kit (Qiagen) according to the manufacturer instructions. For each cell stage and condition analyzed between 0.1 and 1×10⁶ cells were collected for RNA extraction. For qRT-PCR, cDNA synthesis was first performed using the iScript™ cDNA Synthesis Kit (Bio-Rad) and 500 ng of isolated RNA per reaction. qRT-PCR reactions were performed in triplicate with 10 ng of template cDNA per reaction using a CFX96™ Real-Time PCR Detection System and the iQ™ SYBR® Green Supermix (Bio-Rad). PCR of the TATA-binding protein (TBP) coding sequence was used as an internal control and relative expression was quantified via double delta CT analysis. For RNA-seq, stranded, single-end sequencing libraries were constructed from isolated RNA using the TruSeq® Stranded mRNA Library Prep Kit (Illumina®) and library sequencing was performed on either a HiSeq 4000 System (Illumina®) or NovaSeq 6000 System (Illumina®) with single-end reads of either 50 or 75 base pairs (bp). Sequencing was performed by the UCSD IGM core research facility. A complete list of RT-qPCR primer sequences is provided in Table 6.

TABLE 6
Primer sequences used for RT-qPCR

| | |
|---|---|
| PDX1 forward | AAG TCT ACC AAA GCT CAC GCG (SEQ ID NO: 23) |
| PDX1 reverse | GTA GGC GCC GCC TGC (SEQ ID NO: 24) |
| NKX6.1 forward | CTG GCC TGT ACC CCT CAT CA (SEQ ID NO: 25) |
| NKX6.1 reverse | CTT CCC GTC TTT GTC CAA CA (SEQ ID NO: 26) |
| FOXA1 forward | GAA GAT GGA AGG GCA TGA AA (SEQ ID NO: 27) |
| FOXA1 reverse | GCC TGA GTT CAT GTT GCT GA (SEQ ID NO: 28) |
| FOXA2 forward | GGG AGC GGT GAA GAT GGA (SEQ ID NO: 29) |
| FOXA2 reverse | TCA TGT TGC TCA CGG AGG AGT A (SEQ ID NO: 30) |
| GCG forward | AAG CAT TTA CTT TGT GGC TGG ATT (SEQ ID NO: 31) |
| GCG reverse | TGA TCT GGA TTT CTC CTC TGT GTC T (SEQ ID NO: 32) |
| TBP forward | TGT GCA CAG GAG CCA AGA GT (SEQ ID NO: 33) |
| TBP reverse | ATT TTC TTG CTG CCA GTC TGG (SEQ ID NO: 34) |

### RNA-Seq Data Analysis

Reads were mapped to the human genome consensus build (hg19/GRCh37) using the Spliced Transcripts Alignment to a Reference (STAR) aligner version 2.45. Normalized gene expression (fragments per kilobase per million mapped reads; FPKM) for each sequence file was determined using Cufflinks version 2.2.1 with the parameters:-library-type fr-firststrand-max-bundle-frags 10000000. Differential gene expression was determined using DESeq2E. Adjusted P-values<0.05 and fold change 2 were considered significant. For RNA-seq corresponding to cells at the HP stage, one replicate was generated. For all other RNA-seq experiments, replicates from two independent hESC differentiations were generated. Pearson correlations between bam files corresponding to each pair of replicates were calculated and are provided in Table 7.

TABLE 7
Pearson correlation coefficients of RNA-seq replicates

| | | | FOXA1/2 | |
|---|---|---|---|---|
| | Non-transfected | | frameshift | FOXA1/2 exon |
| | control | Non-edited | mutation | deletion |
| | H1 hESCs | H1 hESCs | H1 hESCs | H1 hESCs |
| DE | 0.89 | 0.92 | 0.92 | 0.93 |
| GT | 0.96 | 0.91 | 0.91 | 0.92 |
| PP1 | 0.98 | 0.98 | 0.93 | 0.94 |
| PP2 | 0.99 | 0.96 | 0.83 | 0.90 |

### Assay for Transposase Accessible Chromatin Sequencing (ATAC-Seq)

ATAC-seq⁶¹ was performed on approximately 50,000 nuclei. The samples were permeabilized in cold permabilization buffer (0.2% IGEPAL-CA630 (Sigma, Cat #18896), 1 mM DTT (Sigma, Cat #D9779), Protease inhibitor (Roche, Cat #05056489001), 5% BSA (Sigma, Cat #A7906) in PBS (Thermo Fisher Scientific, Cat #10010-23) for 10 min on the rotator in the cold room and centrifuged for 5 min at 500×g at 4° C. The pellet was resuspended in cold tagmentation buffer (33 mM Tris-acetate (pH=7.8) (Thermo Fisher Scientific, Cat #BP-152), 66 mM K-acetate (Sigma, Cat #P5708), 11 mM Mg-acetate (Sigma, Cat #M2545), 16% DMF (EMD Millipore, Cat #DX1730) in Molecular biology water (Corning, Cat #46000-CM)) and incubated with tagmentation enzyme (Illumina, Cat #FC-121-1030) at 37° C. for 30 min with shaking at 500 rpm. The tagmented DNA was purified using MinElute PCR purification kit (QIAGEN, Cat #28004). Libraries were amplified using NEBNext High-Fidelity 2×PCR Master Mix (NEB, Cat #M0541) with primer extension at 72° C. for 5 min, denaturation at 98° C. for 30 s, followed by 8 cycles of denaturation at 98° C. for 10 s, annealing at 63° C. for 30 s and extension at 72° C. for 60 s. After the purification of amplified libraries using MinElute PCR purification kit (QIAGEN, Cat #28004), double size selection was performed using SPRIselect bead (Beckman Coulter, Cat #B23317) with 0.55× beads and 1.5× to sample volume. Finally, libraries were sequenced on HiSeq4000 (Paired-end 50 cycles, Illumina).

### ATAC-Seq Data Analysis

ATAC-seq reads were mapped to the human genome (hg19/GRCh37) using Burrows-Wheeler Aligner⁵³ (BWA) version 0.7.13, and visualized using the UCSC Genome Browser⁵². SAMtools^{t} was used to remove unmapped, low-quality (q<15), and duplicate reads. MACS2⁶²U version 2.1.4 was used to call peaks, with parameters “shift set to 100 bps, smoothing window of 200 bps” and with “nolambda” and “nomodel” flags on. MACS2 was also used to call ATAC-Seq summits, using the same parameters combined with the “call-summits” flag.

For all ATAC-seq experiments, replicates from two independent hESC differentiations were generated. Bam files for each pair of replicates were merged for downstream analysis using SAMtools, and Pearson correlations between bam files for each individual replicate were calculated over a set of peaks called from the merged bam file. Correlations were performed using the command multiBamSummary from the deepTools2 package⁵⁷ with the “-removeOutliers” flag. Correlations are provided in Table 8.

TABLE 8
Pearson correlation coefficients of ATAC-seq replicates

| | Non-transfected | FOXA1/2 exon | SCRAM | shPDX1 |
|---|---|---|---|---|
| | control | deletion | transduced | transduced |
| | H1 hESCs | H1 hESCs | CyT49 hESCs | CyT49 hESCs |
| GT | 0.99 | 0.98 | N/A | N/A |
| PP2 | 0.97 | 0.99 | 0.99 | 1.00 |

### Hi-C Data Analysis

Hi-C data were processed as previously described⁶³. Read pairs were aligned to the hg19 reference genome separately using BWA-MEM with default parameters⁵³. Specifically, chimeric reads were processed to keep only the 5′ position and reads with low mapping quality (<10) were filtered out. Read pairs were then matched, and Picard tools were then used to remove PCR duplicates. Bam files with alignments were further processed into text format as required by Juicebox tools⁶⁴. Juicebox tools were then applied to generate Hi-C files containing normalized contact matrices. All downstream analysis was based on 10 Kb resolution KR-normalized matrices.

Chromatin loops were identified by comparing each pixel with its local background, as described previously⁶⁵ with some modifications. Specifically, only the donut region around the pixel was compared to model the expected count. Briefly, the KR-normalized contact matrices at 10 Kb resolution were used as input for loop calling. For each pixel, distance-corrected contact frequencies were calculated for each surrounding bin and the average of all surrounding bins. The expected counts were then transformed to raw counts by multiplying the counts with the raw-to-KR normalization factor. The probability of observing raw expected counts was calculated using Poisson distribution. All pixels with P-value<0.01 and distance less than 10 Kb were selected as candidate pixels. Candidate pixels were then filtered to remove pixels without any neighboring candidate pixels since they were likely false positives. Finally, pixels within 20 Kb of each other were collapsed and only the most significant pixel was selected. The collapsed pixels with P-value<1×10⁻⁵ were used as the final list of chromatin loops.

### Single-Cell RNA-Sequencing Library Preparation

Pancreatic progenitor cells at day 11 of differentiation were allowed to settle in microcentrifuge tubes and washed with PBS. Cell aggregates were incubated with Accutase® at 37° C. until a single-cell suspension was obtained. Cells were then resuspended in 1 mL ice-cold flow buffer comprised of 0.2% BSA in PBS and stained with propidium iodide (Sigma, Cat #P4170) to distinguish live cells. 500,000 live cells were collected using a FACSAria™ Fusion Flow Sorter, and 10,000 cells per sample were then loaded onto a 10× Chromium Controller and run using Next GEM Single-Cell 3′ v3.1 reagents. Library preparation was performed according to manufacturer's instructions, and libraries were sequenced using a NovaSeq S4 (Paired-end 100 bp reads, Illumina).

### Single-Cell RNA-Sequencing Data Analysis

Sequencing reads were processed using CellRanger⁶⁶ version 6.0.0, and matrices generated by CellRanger were imported into Seurat⁶⁷ version 3 for further processing. Doublet cells (>8000 total features for control cells and >6000 total features for motif optimized cells), low-coverage cells (<3000 total features for control cells and <2500 total features for motif optimized cells), and poor-quality cells (>10% mitochondrial reads for both conditions) were removed from further analysis. Each dataset was Log Normalized with a scale factor of 10,000 using the command “NormalizeData.” Percentage of mitochondrial genes were regressed out of each dataset using the command “ScaleData.” Integration anchors for each dataset were identified using “FindlntegrationAnchors,” and datasets were integrated using the command “IntegrateData.” Principal component analysis was performed for the integrated dataset using the command “RunPCA,” and UMAP plots were generated through “RunUMAP.” Clusters were defined running the commands “FindNeighbors” and “FindClusters” at a resolution of 0.03, and marker genes were identified using “FindMarkers.” Feature plots and dot plots were generated using the commands “Featureplot” and “Dotplot,” and differential expression of genes co-expressed with NKX6.1 was calculated by subsetting for cells expressing NKX6.1 and using “FindMarkers” to determine differential genes between control and motif optimized cells. Wilcoxon rank sum tests were used to calculate differential expression.

### Gene Ontology Analysis

Gene ontology analysis for enhancer groups was performed using GREAT⁶⁸ version 4.0.4 with the default parameters. Gene ontology for differentially expressed genes and genes associated with class I and class II enhancers was performed using Metascape⁶⁹ using default parameters.

### Identification of Super-Enhancers

To define pancreatic super-enhancers, we first identified pancreatic enhancers as distal genomic regions exhibiting a ≥2-fold increase in H3K27ac ChIP-seq signal during pancreas induction. We then used Rank Ordering of Super-enhancers (ROSE) softwarez^{21, 70} to join identified pancreatic enhancers within a 12.5 kb span and rank these joined enhancers based on intensity of H3K27ac ChIP-seq signal. These joined enhancers were plotted based on H3K27ac signal, and pancreatic super-enhancers were defined as joined enhancers ranking above the inflection point of the resulting graph.

### Principal Component Analysis

For RNA-seq data, transcriptomes were first filtered for genes expressed (FPKM 1) in at least one condition, then log 10 transformed. For distal H3K27ac signals, H3K27ac peaks were filtered for distal enhancers (≥2.5 kb from any annotated TSS). Based on filtered values, PCA plots were generated using the PRComp package in R.

### Quantification of Changes in H3K27ac Signal

HOMER⁵⁵ was used to annotate raw H3K27ac ChIP-seq reads over distal enhancers at developmental stages both before and after lineage induction. HOMER was then used to invoke the R package DESeq2⁶⁰ version 3.10 for differential analysis, using default parameters.

### Quantification of Changes in TF ChIP-Seq and ATAC-Seq Signal

HOMER⁵⁵ was used to annotate raw FOXA1 and FOXA2 ChIP-seq reads, as well as ATAC-seq reads over PDX1-bound class I and class II enhancers in cells transfected with SCRAM and shPDX1 lentivirus. HOMER was then used to invoke the R package DESeq2⁶⁰ for differential analysis, using the flag “norm2total.”

### Assignment of Enhancer Target Genes

RNA-seq data were filtered for expressed genes (FPKM 1) at the PP2 stage, and BEDTools⁵⁶ “closest” command was used to assign each enhancer to the nearest annotated TSS.

### Motif Enrichment Analysis

HOMER⁵⁵ was used for comparative motif enrichment analyses, using the command findMotifsGenome.pl. de novo motifs were assigned to TFs based on suggestions generated by HOMER.

### Identification of FOXA Motifs and Generation of Log-Odds Scores

FOXA1 and FOXA2 PWMs were selected to encompass the most divergent PWMs for each TF. PWMs were downloaded from the JASPAR database²⁷, and occurrences with associated log-odds scores were quantified using the FIMO feature within the MEMEsuit package⁷¹ version 5.1.1.

### Calculation of Positional Motif Enrichment

Identified ATAC-seq summits on class I and class II enhancers were flanked by 500 bp in each direction, and the CENTRIMO feature within the MEMEsuit package⁷² version 5.1.1 was used to determine enrichment at summits for selected PWMs associated with FOXA1 and FOXA2, as well as to graph the positional probability of motif occurrence with respect to ATAC-seq summits.

### ATAC-Seq Footprinting Analysis

ATAC-seq footprinting was performed as previously described⁷³. In brief, diploid genomes for CyT49 were created using vcf2diploid (version 0.2.6a)⁷⁴ and genotypes called from whole genome sequencing and scanned for a compiled database of TF sequence motifs from JASPAR⁷⁵ and ENCODE⁷⁶ with FIMO (version 4.12.0)⁷¹ using default parameters for p-value threshold and a 40.9% GC content based on the hg19 human reference genome. Footprints within ATAC-seq peaks were discovered with CENTIPEDE (version 1.2)⁷⁷ using cut-site matrices containing Tn5 integration counts within a ±100 bp window around each motif occurrence. Footprints were defined as those with a posterior probability ≥0.99.

### Permutation-Based Significance

A random sampling approach (10,000 iterations) was used to obtain null distributions for enrichment analyses, in order to obtain P-values. Null distributions for enrichments were obtained by randomly shuffling enhancer regions using BEDTools⁵⁶ and overlapping with FOXA1/2-binding sites. P-values<0.05 were considered significant.

### Quantification and Statistical Analysis

Statistical analyses were performed using GraphPad Prism (v8.1.2), and R (v3.6.1). Statistical parameters such as the value of n, mean, standard deviation (SD), standard error of the mean (SEM), significance level (n.s., not significant; *P<0.05; **P<0.01; and ***P<0.001), and the statistical tests used are reported in the figures and figure legends. Unless otherwise noted, the “n” refers to the number of independent hESC differentiation experiments analyzed (biological replicates). All bar graphs and line graphs are displayed as mean±S.E.M, and all box plots are centered on median, with box encompassing 25th-75th percentile and whiskers extending up to 1.5 interquartile range. Statistically significant gene expression changes were determined with DESeq2⁶⁰, and significantly enriched gene ontology terms were identified using Metascape⁶⁹.

For all bar graphs of gene expression measured via qPCR, each plotted point represents the average of three technical replicates. For all immunofluorescence, representative images are shown from n≥2 independent differentiations. For all flow cytometry analyses, representative plots are shown from n=3 independent differentiations.

### Example 2

### FOXA1 and FOXA2 are Necessary for Pancreatic Lineage Induction

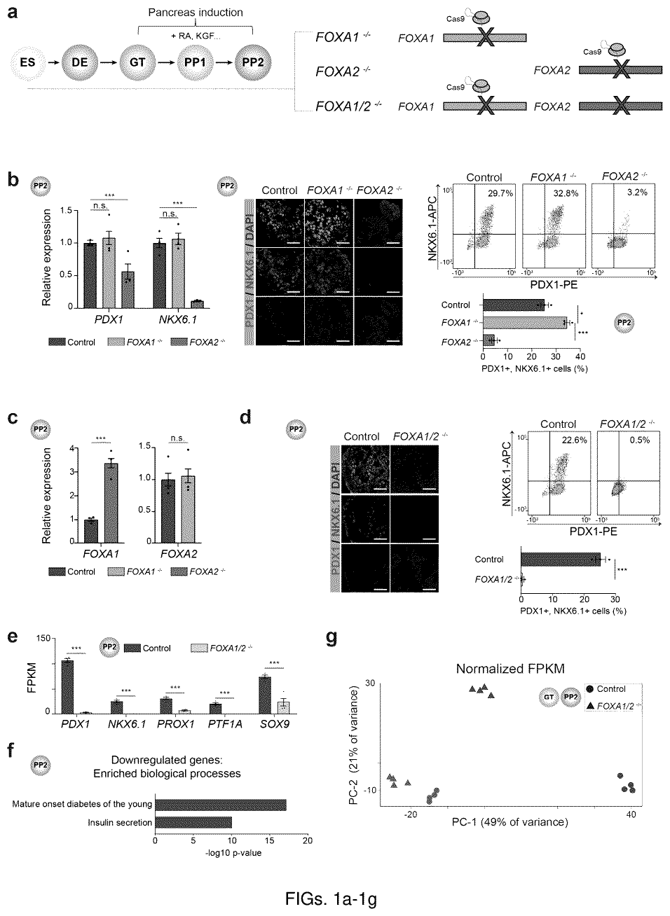

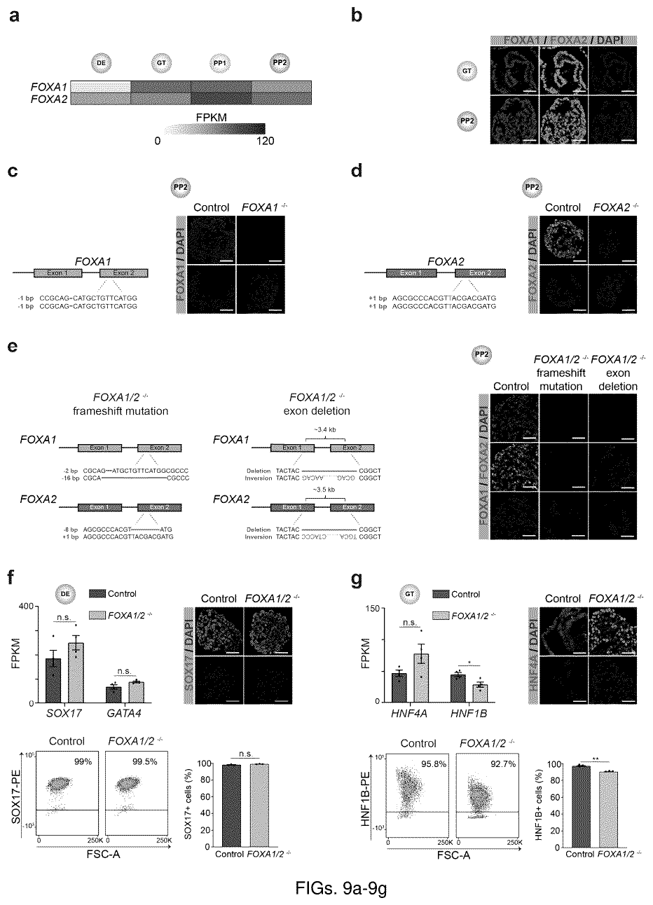

To investigate the role of FOXA1/2 in pancreas development, we employed a hPSC differentiation protocol in which cells transition stepwise to the pancreatic fate through sequential exposure to developmental signaling cues (FIG. 1a). The pancreatic lineage is induced by retinoic acid from gut tube (GT) intermediates, resulting in expression of the pancreatic markers PDX1 in early pancreatic progenitors (PP1) and NKX6.1 in late pancreatic progenitors (PP2). FOXA1 and FOXA2 were expressed from the definitive endoderm (DE) stage onwards (FIGS. 9a and 9b), and levels of FOXA1 and FOXA2 were similar in GT, PP1, and PP2 (FIG. 9a).

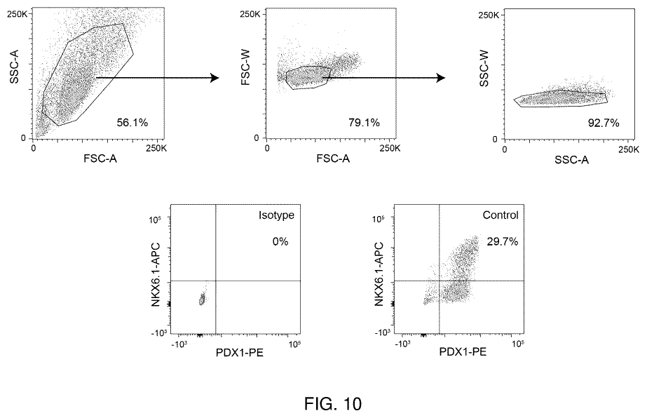

To determine a possible requirement for FOXA1 and FOXA2 in pancreas development, we deleted FOXA1 or FOXA2 in human embryonic stem cells (hESCs) (FIG. 1a and FIGS. 9c and 9d) and differentiated control, FOXA1^{-/-}, and FOXA2^{-/-} hESC lines into pancreatic progenitors. Analysis of PDX1 and NKX6.1 expression revealed a requirement for FOXA2 but not FOXA1 for pancreatic lineage induction (FIG. 1b and FIG. 10), consistent with recent findings³. The presence of residual PDX1⁺ and NKX6.1+ cells and increased FOXA1 levels in FOXA2^{-/-} pancreatic progenitors (FIGS. 1b and 1c) suggests FOXA1 partially compensates for FOXA2 deficiency. Therefore, FOXA1^{-/-}; FOXA2^{-/-} (FOXA1/2^{-/-}) hESC lines were generated (FIG. 9e) and analyzed phenotypes at the DE, GT, and PP2 stages. At the DE and GT stages, similar numbers of FOXA1/2^{-/-} and control cells expressed the DE marker SOX17 and GT marker HNF1B, respectively (FIGS. 9f and 9g). In contrast, pancreas induction was blocked in FOXA1/2^{-/-} cells, as evidenced by an almost complete absence of PDX1⁺ and NKX6.1⁺ cells, reduced expression of early pancreatic TFs, and down-regulation (≥2-fold change, FDR≤0.05) of genes associated with pancreas-specific biological processes (FIGS. 1d-1f). Principal component analysis (PCA) of transcriptome data further confirmed that FOXA1/2^{-/-} and control cells were more similar at the GT stage than at the PP2 stage (FIG. 1g). Together, these findings show that FOXA1 and FOXA2 control pancreatic lineage induction from gut tube lineage intermediates in a partially redundant manner.

### Example 3

### FOXA Transcription Factors Exhibit Two Temporal Patterns of Recruitment to Pancreatic Enhancers

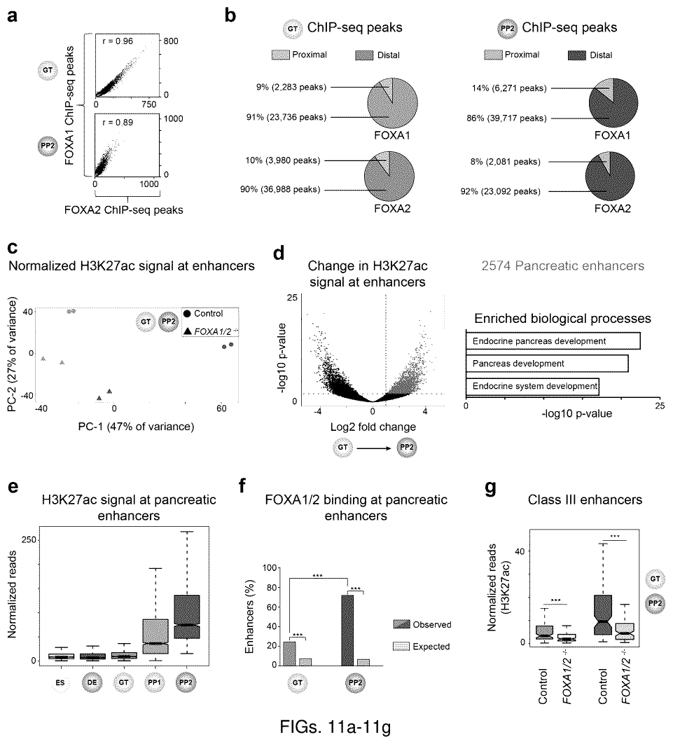

To identify transcriptional targets of FOXA1/2 during pancreatic lineage induction, we mapped FOXA1/2-binding sites at the GT and PP2 stages. Consistent with the partial functional redundancy between FOXA1 and FOXA2 (FIGS. 1b-1d), FOXA1 and FOXA2-binding sites were highly correlated at both stages (FIG. 11a). FOXA1/2 mostly bound to distal sites (>2.5 kb from TSS; FIG. 11b), suggesting regulation of enhancers by FOXA1/2. To test this, GT and PP2 enhancers were defined as distal H3K27ac peaks (>2.5 kb from TSS) and enhancer activity was compared based on H3K27ac signal in control and FOXA1/2^{-/-} cells at the GT and the PP2 stages. Like gene expression (FIG. 1g), H3K27ac profiles in FOXA1/2^{-/-} and control cells differed more substantially at the PP2 than at the GT stage (FIG. 11c), showing that FOXA1/2 deletion has broad impact on regulation of enhancer activity during the GT to PP2 transition.

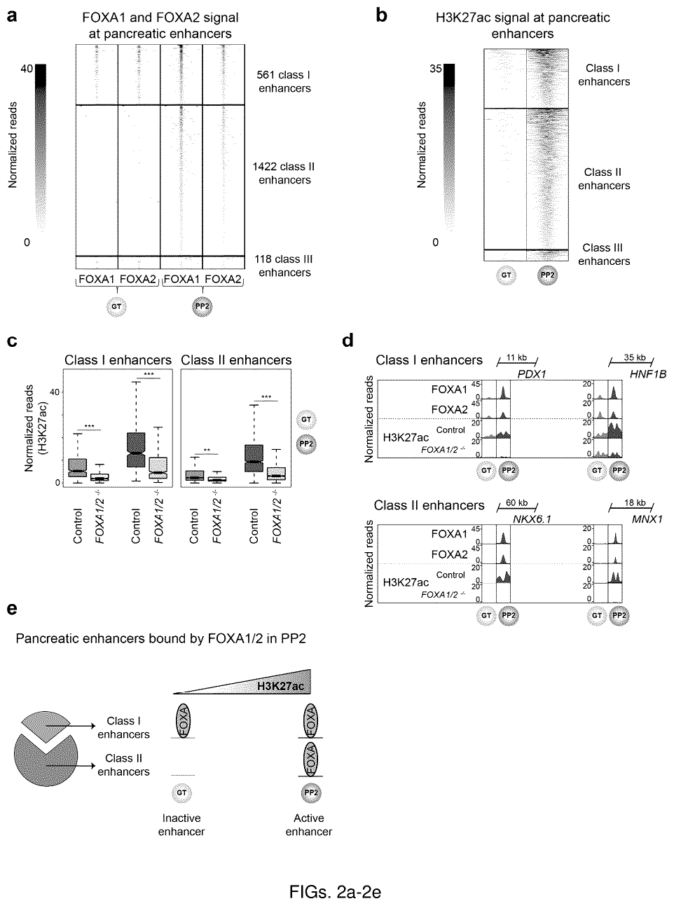

To investigate specific mechanisms by which FOXA1/2 mediates pancreatic lineage induction, all FOXA1/2-bound pancreatic enhancers were identified that are activated upon pancreatic lineage induction. To this end, enhancers that exhibited a 2-fold increase in H3K27ac signal were first identified from the GT to the PP2 stage (2574 enhancers, hereafter referred to as pancreatic enhancers; FIGS. 11d and 11e). As expected, genes near these enhancers were predicted to regulate biological processes associated with pancreas development. Second, FOXA1/2 binding was analyzed at these pancreatic enhancers, revealing that 72% were FOXA1/2-bound at the PP2 stage (FIG. 11f). Consistent with prior reports^{1, 3}, FOXA1/2 occupancy was observed at the GT stage preceding pancreatic lineage induction. Surprisingly, however, the percentage of pancreatic enhancers bound by FOXA1/2 was significantly lower at the GT compared to the PP2 stage, implying that not all pancreatic enhancers engage FOXA1/2 before lineage induction. To comprehensively characterize temporal patterns of FOXA1/2 recruitment, all pancreatic enhancers with FOXA1 or FOXA2 binding at the GT and/or PP2 stages were identified and FOXA1/2 ChIP-seq signal was quantified at these sites (FIG. 2a). three distinct patterns of FOXA1/2 occupancy were observed: class I enhancers (561) were bound by FOXA1/2 at both the GT and PP2 stages, class II enhancers (1422) were FOXA1/2-bound only at the PP2 stage, and the overall small group of class III enhancers (118) was FOXA1/2-bound only at the GT stage (FIG. 2a). Analysis of H3K27ac signal intensity at the GT and PP2 stages showed similar patterns of H3K27ac signal at all enhancers (FIG. 2b), suggesting that enhancers of all classes are mostly inactive at the GT stage and become activated during pancreatic lineage induction. Activation of enhancers of all classes during the GT to PP2 transition was dependent on FOXA1/2 (FIG. 2c and FIG. 11g). Since the predominant patterns were either maintenance of FOXA1/2 binding (class I) or de novo FOXA1/2 occupancy (class II) after pancreas induction, class III enhancers were excluded from further analyses. Examples of both class I and class II enhancers were identified in proximity to gene bodies of pancreatic lineage-determining TFs, such as PDX1, HNF1B, NKX6.1, and MNX1 (FIG. 2d). Consistent with the H3K27ac pattern, the PDX1 class I enhancer and the NKX6.1 class II enhancer are both inactive in GT and active in PP2 in enhancer reporter assays¹. Together, this analysis shows that FOXA1/2 recruitment to pancreatic enhancers precedes lineage induction at only a small subset of enhancers, while FOXA1/2 recruitment to most pancreatic enhancers coincides with lineage induction (FIG. 2e).

### Example 4

### Primed and Unprimed Pancreatic Enhancers Reside in Distinct Regulatory Domains

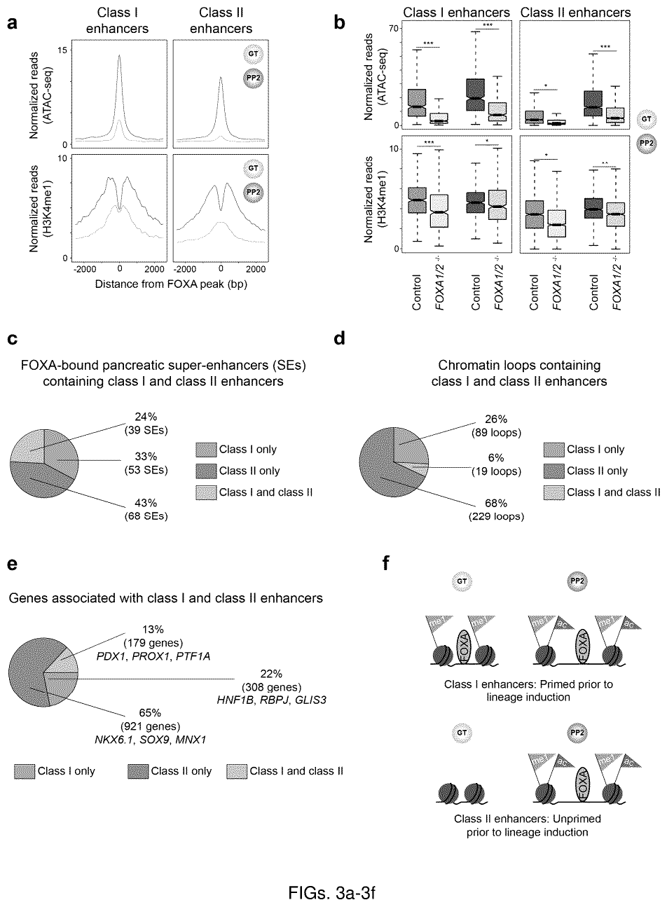

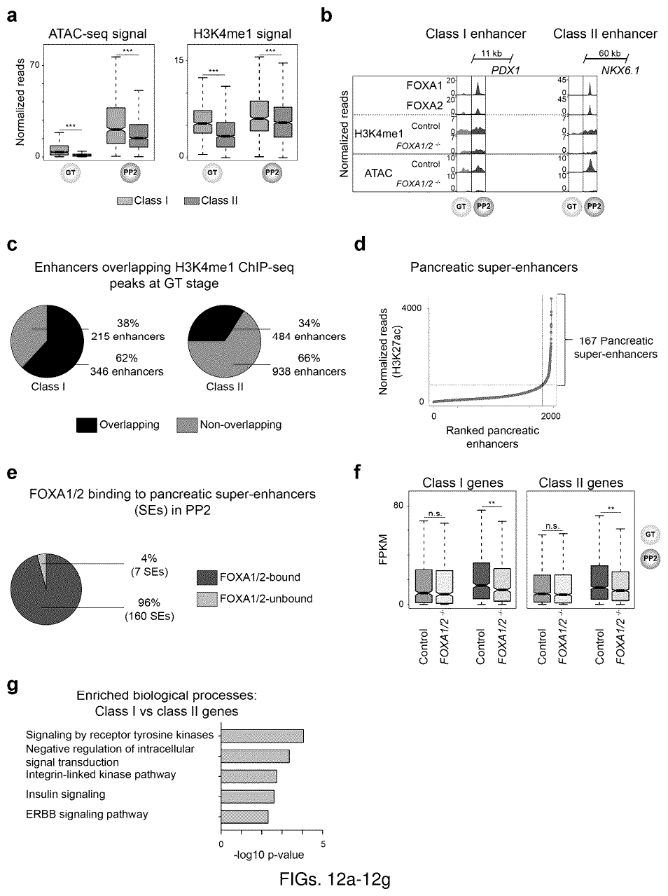

Given early recruitment of FOXA1/2 to class I but not class II enhancers, we hypothesized that the two classes could differ in their temporal pattern of gain in chromatin accessibility and H3K4me1 deposition, predicting that early FOXA1/2 occupancy at class I enhancers would lead to chromatin priming. As predicted, class I enhancers exhibited open chromatin and H3K4me1 deposition at the GT stage (FIG. 3a and FIGS. 12a and 12b). By contrast, class II enhancers acquired these features largely with pancreatic lineage induction (FIG. 3a and FIGS. 12a and 12b), identifying primed chromatin as a feature of class I enhancers. Although a subset of class II enhancers was marked by H3K4me1 at the GT stage, this population comprised the minority of class II enhancers (FIG. 12c). At both class I and class II enhancers, H3K4me1 deposition and gain in chromatin accessibility during lineage induction was FOXA1/2-dependent (FIG. 3b and FIG. 12b), demonstrating that FOXA1/2 are necessary for chromatin remodeling at both classes of enhancers.

To determine whether class I and class II enhancers function together within larger regions of active chromatin such as super-enhancers-, or whether they reside in distinct regulatory domains, to distinguish between these possibilities, 167 super-enhancers were defined among the 2574 pancreatic enhancers identified in FIG. 11d (FIG. 12d) and it was found that 160 (96%) were FOXA1/2-bound at the PP2 stage (FIG. 12e). Analysis of overlap between class I or class II enhancers and FOXA-bound super-enhancers revealed that most FOXA-bound super-enhancers (76%) contained either class I or class II enhancers but not both (FIG. 3c). Furthermore, Hi-C datasets produced from PP2 stage cells were analyzed and it was found that class I and class II enhancers were mostly located in non-overlapping 3D chromatin loops (FIG. 3d). This evidence indicates that class I and class II enhancers reside largely within distinct gene regulatory domains and therefore likely function independently.

To identify target genes of class I and class II enhancers, enhancers were assigned to their nearest expressed gene at the PP2 stage, and predictions were validated by showing regulation of these genes by FOXA1/2 (FIG. 12f). Consistent with their location in distinct regulatory domains (FIGS. 3c and 3d), class I and class II enhancers mostly associated with distinct genes, including pancreatic lineage-determining TFs (FIG. 3e). Of note, gene ontology analysis of genes regulated by class I compared to class II enhancers revealed roles for class I enhancer-associated genes in cellular signal transduction pathways (FIG. 12g), whereas no comparative enrichment of specific gene ontology terms was observed for class II enhancer-associated genes. Together, these results suggest that two distinct mechanisms establish the pancreatic gene expression program: a subset of pancreatic genes is regulated by enhancers that undergo FOXA1/2-mediated chromatin priming at the gut tube stage, whereas most pancreatic genes are regulated by enhancers that are unprimed prior to pancreatic lineage induction, and to which FOXA1/2 are recruited upon lineage induction (FIG. 3f).

### Example 5

### Distinct DNA Sequence Motifs at Primed and Unprimed Pancreatic Enhancers

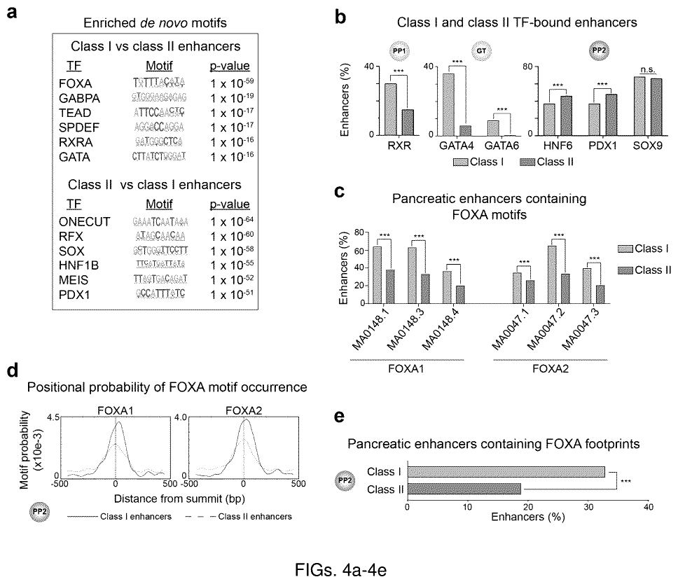

Next, mechanisms that could explain the observed temporal differences in FOXA1/2 binding to class I (primed) and class II (unprimed) pancreatic enhancers were investigated. To test whether differences in DNA sequence could provide an explanation, de novo motif analysis was conducted to identify motifs enriched at class I enhancers against a background of class II enhancers. Class I enhancers were enriched for FOXA motifs and motifs for several signal-dependent TFs, including the ETS family TFs GABPA and SPDEF, the downstream effector of Hippo signaling TEAD, and the retinoic acid receptor RXRA (FIG. 4a). Work in model organisms has identified critical roles for ETS TFs as well as Hippo and retinoic acid signaling in early pancreatic development¹, suggesting that pancreatic lineage-inductive signals are read at class I enhancers by partnering of FOXA1/2 with signal-dependent TFs. ChIP-seq analysis for RXR confirmed preferential RXR binding to class I compared to class II enhancers at the PP1 stage (FIG. 4b). Class I enhancers were also enriched for GATA TF motifs (FIG. 4a), and a higher percentage of class I than class II enhancers bound GATA4 and GATA6 at the GT stage (FIG. 4b). Given that GATA TFs cooperatively bind with FOXA1/2 to DNA¹⁷, GATA4/6 could facilitate FOXA1/2 recruitment to a subset of class I enhancers prior to pancreas induction.

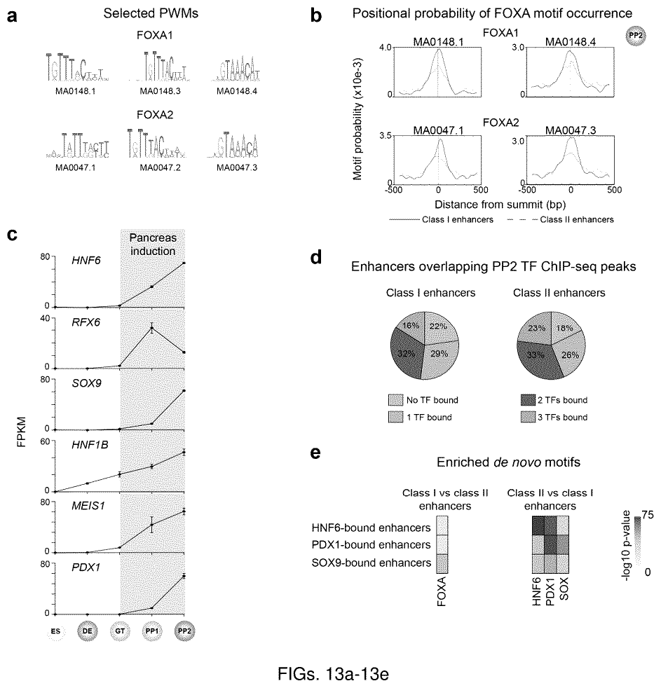

Since FOXA1/2 binding to class I enhancers precedes binding to class II enhancers (FIG. 2a) and FOXA motifs are enriched at class I compared to class II enhancers (FIG. 4a), different mechanisms could underlie FOXA1/2 recruitment to the two classes of enhancers. Binding site selection of pioneer TFs such as FOXA1/2 has been shown to depend on motif abundance, strength, and position^{17, 18, 19, 26}. Therefore, FOXA motifs at class I and class II enhancers were analyzed for these features. To determine abundance and strength of FOXA motifs, position-weighted matrices (PWMs) corresponding to three FOXA1 and three FOXA2 motifs were selected from JASPAR²⁷ (FIG. 13a), occurrences of each motif at class I and class II enhancers were identified, and a log-odds score was generated to measure how closely the DNA sequence at each identified motif occurrence matched the PWM. Class I enhancers were significantly enriched for occurrences of all six FOXA motifs compared to class II enhancers (FIG. 4c). Furthermore, three of the FOXA motifs had significantly higher log-odds scores at class I than class II enhancer occurrences (MA0047.2, MA0148.1, and MA0148.3; P=1.54×10⁻², 1.10×10⁻³, and 1.03×10⁻², respectively; Wilcoxon rank sum test). Thus, class II enhancers contain more degenerate and fewer FOXA motifs compared to class I enhancers. The positioning of FOXA motifs relative to open chromatin was additionally examined by identifying regions of greatest chromatin accessibility at class I and class II enhancers in PP2 stage cells (n=531 and n=1257 ATAC-seq summits in class I and class II enhancers, respectively) and enrichment of each FOXA motif at these regions was determined. Occurrence of all FOXA motifs was enriched at ATAC-seq summits at class I compared to class II enhancers (FIG. 4d and FIG. 13b), indicating that regions of greatest chromatin accessibility at class I enhancers are more likely to harbor FOXA motifs. ATAC-seq footprinting analysis further revealed a higher occurrence of FOXA footprints at class I than at class II enhancers (FIG. 4e), indicative of either longer FOXA1/2 DNA residence times or more direct interaction of FOXA1/2 with DNA at class I enhancers&. Together, this analysis reveals features of FOXA motifs at class I pancreatic enhancers previously associated with canonical FOXA1/2 pioneer TF activity^{17, 18}.

To further elucidate differences in mechanisms of FOXA recruitment to class I and class II enhancers, de novo motifs enriched at class II enhancers were identified against a background of class I enhancers. Here, enrichment of motifs was observed for pancreatic lineage-determining TFs, such as ONECUT (HNF6), SOX (SOX9), HNF11B, and PDX1 (FIG. 4a), which sharply increased in expression during pancreatic lineage induction (FIG. 13c). To determine whether these TFs exhibit preferential binding to class II enhancers, HNF6, PDX1, and SOX9-binding sites were mapped genome-wide at the PP2 stage (FIG. 4b and FIG. 13d). Overall, it was found that similar percentages of class I and class II enhancers were bound by HNF6, PDX1, and SOX9 at the PP2 stage (FIG. 4b). To determine whether the difference in sequence motif enrichment between class I and class II enhancers is also observed when focusing on enhancers bound by a specific TF, motifs at HNF6-, PDX1-, or SOX9-bound enhancers were analyzed. Still, class I enhancers were enriched for FOXA and class II enhancers for ONECUT (HNF6), PDX1, and SOX motifs (FIG. 13e). Thus, despite differences in DNA sequence motifs between primed (class I) and unprimed (class II) enhancers, both classes of enhancers are occupied by FOXA1/2, as well as pancreatic lineage-determining TFs after pancreatic lineage induction.

### Example 6

### FOXA1/2 Binding to a Subset of Unprimed Enhancers Depends on PDX1

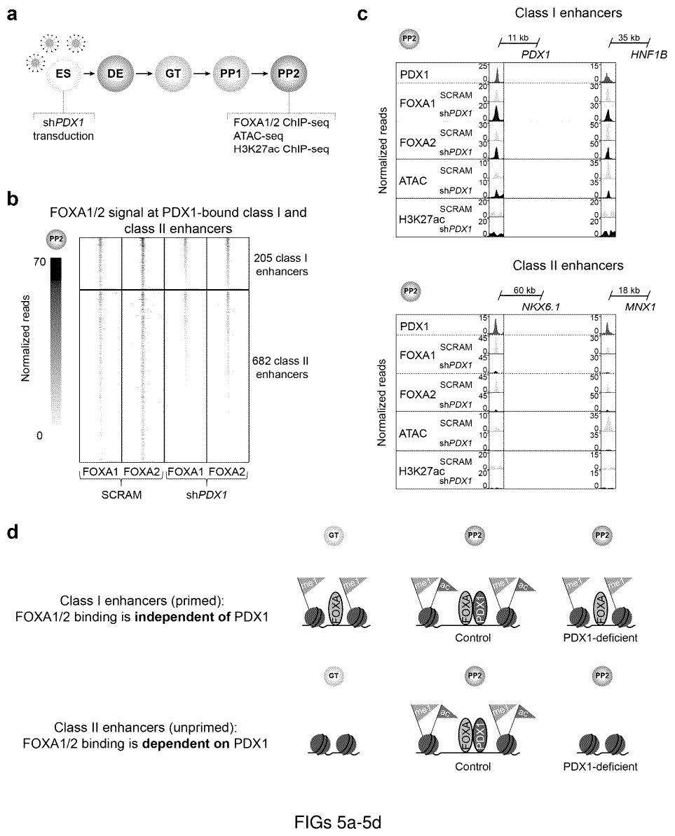

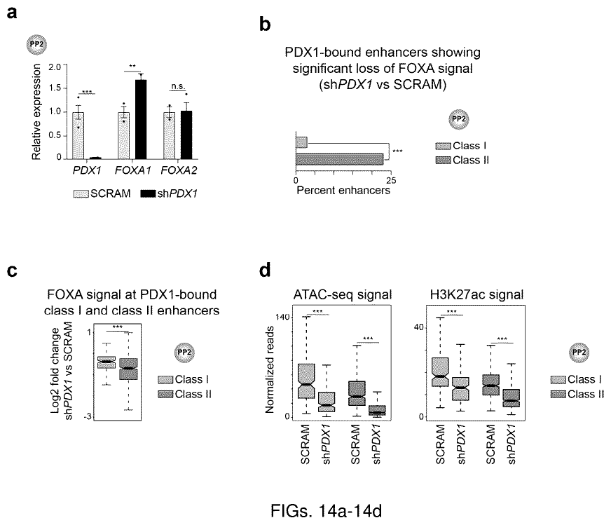

Since motifs for pancreatic lineage-determining TFs, such as PDX1, were enriched at class II compared to class I enhancers (FIG. 4a), FOXA1/2 recruitment to class II enhancers could require cooperativity with lineage-determining TFs. To test this, FOXA1/2 binding, chromatin accessibility, and H3K27ac signal were analyzed in PDX1-deficient pancreatic progenitors (FIG. 5a and FIG. 14a). Focusing on PDX1-bound enhancers (n=205 class I enhancers and 682 class II enhancers), it was found that loss of PDX1 reduced FOXA1/2 binding to a greater extent at class II than class I enhancers (FIG. 5b and FIGS. 14b and 14c), exemplified by class I enhancers near PDX1 and HNF1B, and class II enhancers near NKX6.1 and MNX1 (FIG. 5c). In total, 23% of PDX1-bound class II enhancers exhibited a significant loss (≥2-fold decrease, P. adj.<0.05) in FOXA1/2 ChIP-seq signal after PDX1 knock-down compared to only 3% of PDX1-bound class I enhancers (FIG. 14b). Furthermore, PDX1-bound class II enhancers showed greater loss of FOXA1/2 signal than PDX1-bound class I enhancers (FIG. 14c). Given substantial overlap between binding sites for pancreatic lineage-determining TFs (FIG. 13d), it is possible that other TFs recruit FOXA1/2 to PDX1-bound class II enhancers where FOXA1/2 occupancy is not significantly affected. Loss of PDX1 led to a significant reduction in ATAC-seq and H3K27ac signal at both class I and class II enhancers (FIG. 14d), showing that full acquisition of chromatin accessibility and enhancer activation during pancreas induction require PDX1 at primed and unprimed enhancers.

Collectively, these findings show that despite similar mechanisms for their activation, primed and unprimed pancreatic enhancers differ in sequence logic and mechanism of FOXA1/2 recruitment (FIG. 5d). Primed enhancers have abundant and strong FOXA motifs, and FOXA1/2 are recruited to primed enhancers prior to pancreatic lineage induction largely independent of the pancreatic TF PDX1. By contrast, unprimed enhancers have fewer and weaker FOXA motifs, and a proportion of unprimed enhancers requires PDX1 for FOXA1/2 recruitment.

### Example 7

### Altering FOXA Motif Strength Redefines Temporal FOXA1/2-Binding Patterns

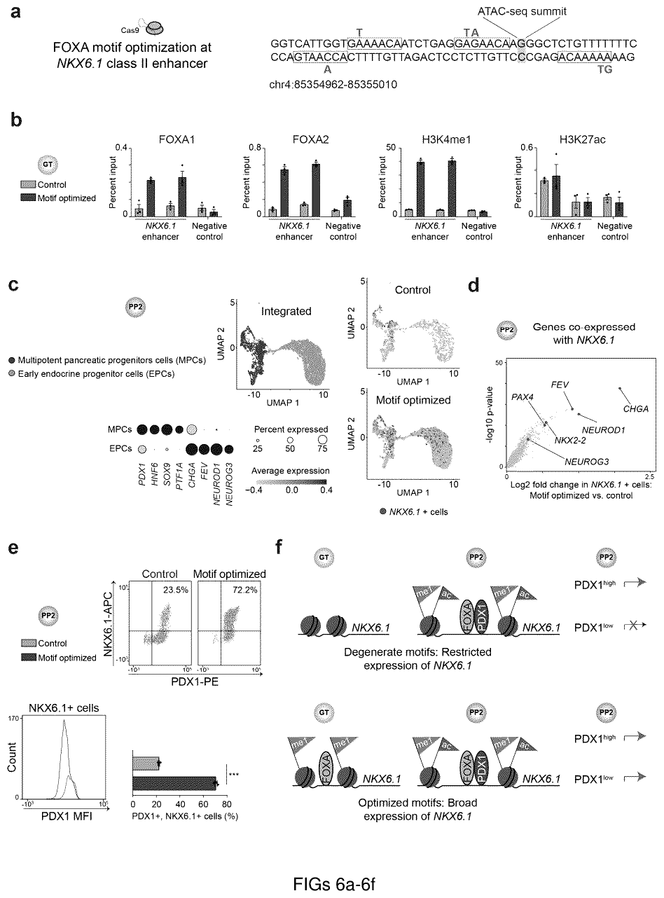

To determine the extent to which the timing and mechanism of FOXA1/2 recruitment are solely dependent on DNA sequence, and since stronger FOXA motifs are a characteristic of class I enhancers, FOXA motifs were optimized at a class II enhancer via CRISPR-Cas9 genome editing and mapping FOXA1/2 binding. For this, an unprimed class II enhancer near NKX6.1 was selected for editing in hESCs. This enhancer lacks FOXA1/2 binding (FIG. 2d), accessible chromatin (FIG. 12b), and H3K4me1 signal (FIG. 12b) prior to pancreas induction. Furthermore, in the absence of PDX1, FOXA1/2 do not bind to this enhancer (FIG. 5c). Examination of the NKX6.1 enhancer revealed four degenerate FOXA motifs surrounding the ATAC-seq summit (FIG. 6a). Six base pairs were altered within the enhancer to strengthen the FOXA motifs (referred to as motif optimized) (FIG. 6a). Optimizing FOXA motifs resulted in FOXA1/2 recruitment to the NKX6.1 enhancer at the GT stage prior to pancreas induction (FIG. 6b). Early FOXA1/2 recruitment was accompanied by H3K4me1 but not H3K27ac deposition at the GT stage (FIG. 6b), supporting that FOXA1/2 prime enhancers prior to activation. Thus, optimization of FOXA-binding motifs is sufficient to convert an unprimed class II enhancer into a primed class I enhancer.

### Example 8

### Optimizing FOXA Motifs Broadens the Domain of Target Gene Expression

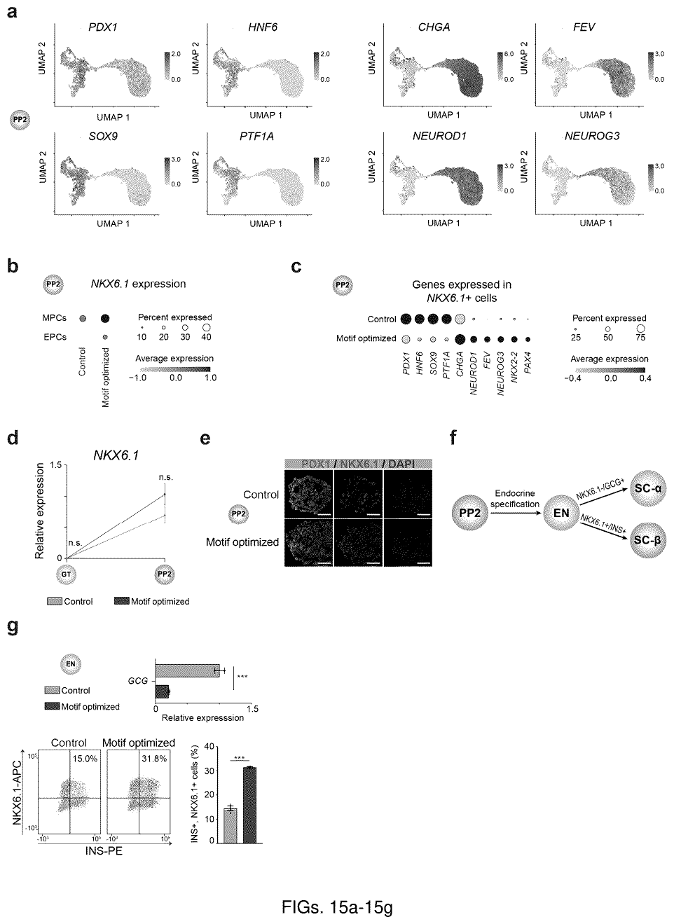

To define the relationship between FOXA motif strength and NKX6.1 target gene expression, single-cell RNA-sequencing of PP2 cells was conducted from control and motif optimized cell lines. Consistent with prior studies²⁹, it was observed a population of multipotent pancreatic progenitor cells expressing high levels of pancreatic lineage-determining TFs (e.g., PDX1, HNF6, SOX9, and PTF1A), as well as a population of early endocrine progenitor cells expressing endocrine TFs and genes (e.g., NEUROG3, NEUROD1, FEV, and CHGA) but lower levels of PDX1 (FIG. 6c and FIG. 15a). In control PP2 cultures, NKX6.1 expression was restricted to multipotent pancreatic progenitors with high PDX1 expression. By contrast, NKX6.1 was broadly expressed in motif optimized cultures, including in cells expressing lower levels of PDX1 (FIGS. 6c and 6d and FIGS. 15b and 15c). Consistent with the lack of enhancer activation in motif optimized GT stage cells (FIG. 6b), there was no premature expression of NKX6.1 at the GT stage (FIG. 15d). These findings indicate that optimizing FOXA motif strength renders NKX6.1 expression independent of high levels of PDX1. Corroborating this conclusion, it was found NKX6.1 protein restricted to progenitors with high levels of PDX1 in control cultures, whereas motif optimized cultures contained a population of NKX6.1+/PDX1^{low} cells (FIG. 6e and FIG. 15e). In sum, these findings show that increasing FOXA motif strength is sufficient to allow for FOXA recruitment independent of cooperative interactions with pancreatic lineage-determining TFs and that converting an unprimed into a primed enhancer lowers the target gene expression threshold (FIG. 6f).

Given that alpha cells are derived from NKX6.1⁻ endocrine progenitors, whereas beta cells arise from NKX6.1⁺ endocrine progenitors³⁰, effects of broader NKX6.1 expression among progenitors on cell fate allocation were examined. To this end, motif optimized and control cells were differentiated to the early endocrine cell stage, when pre-alpha and pre-beta cells can be distinguished²⁹ (FIG. 15f). It was observed a two-fold increase in NKX6.1⁺/insulin⁺ cells accompanied by a decrease in glucagon expression (FIG. 15g), suggesting a pre-alpha to a pre-beta cell fate shift. These results suggest that barriers to enhancer activation and target gene expression imposed by DNA sequence at class II enhancers are biologically relevant for cell lineage allocation during development.

### Example 9

### Distinct Temporal Patterns of FOXA1/2 Occupancy Distinguish Hepatic and Alveolar Enhancers

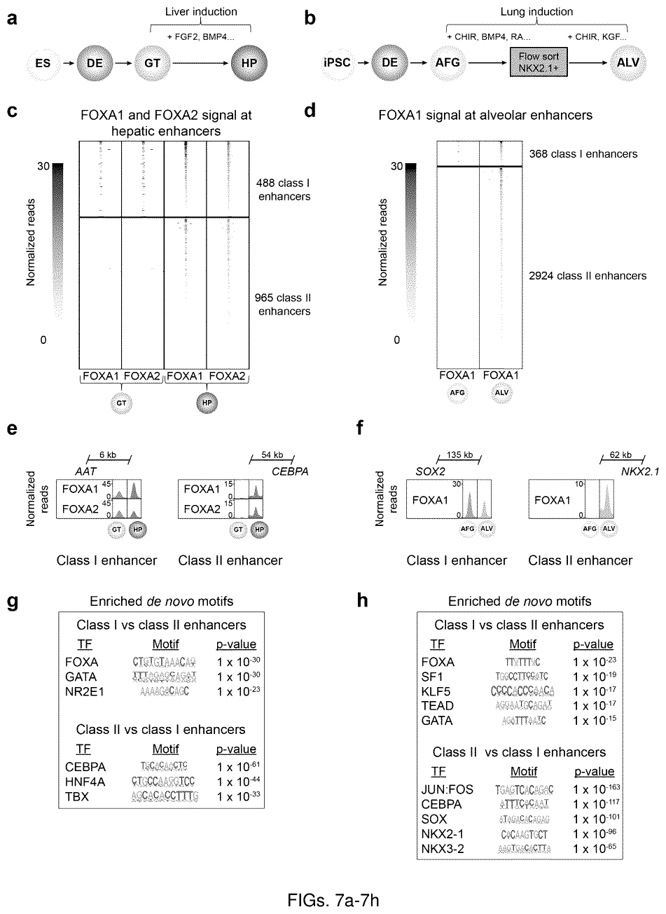

To determine whether the identified mechanisms of enhancer activation during organ development are universal across endodermal lineages, liver and lung enhancers, which like pancreatic enhancers undergo chromatin priming in gut endoderm¹ were also analyzed. Like pancreas development, both early liver and lung development depend on FOXA TFs^{4, 5, 6}. Furthermore, previous studies have demonstrated FOXA binding to primed liver enhancers in gut endoderm prior to organ lineage induction^{1, 11}. To test whether class I and class II enhancers can be distinguished during liver and lung development, the hepatic fate from hESC-GT stage intermediates was induced (FIG. 7a), and distal lung alveolar epithelial type 2-like cells (iAT2s) grown at 95% purity as 3D alveolospheres (ALV) from iPSCs were generated (FIG. 7b)^{1, 31}. For liver, H3K27ac signal and FOXA1/2 binding before liver induction at the GT stage and in hepatic progenitors (HP) were analyzed. For lung, H3K27ac signal and FOXA1 binding before lung induction in anteriorized foregut (AFG) and at the ALV stage were analyzed.

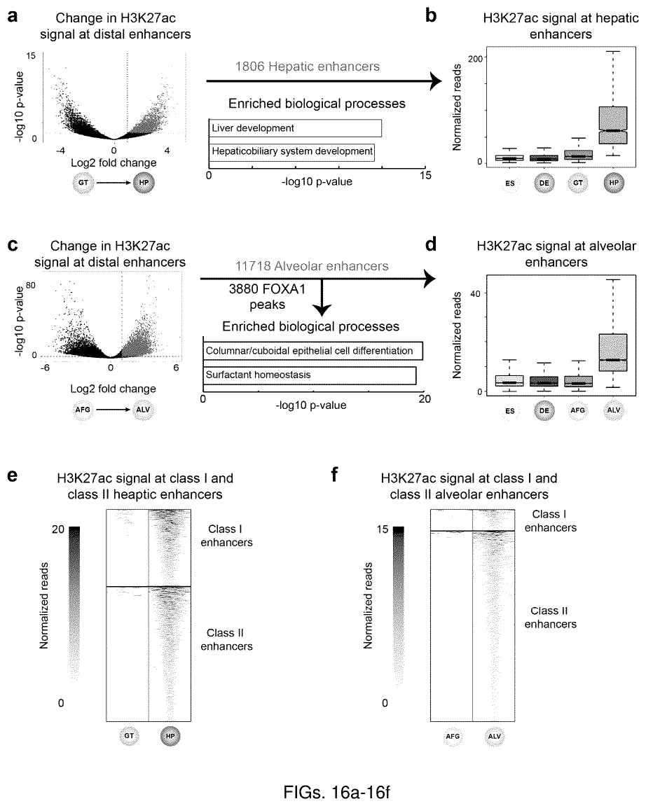

Analogous to the strategy used for identifying pancreatic enhancers (FIG. 11d), hepatic and alveolar enhancers were identified based on gain in H3K27ac signal during the GT to HP and AFG to ALV transitions, respectively (≥2-fold change in H3K27ac, FDR≥0.05; FIGS. 16a-16d). Subsequently, FOXA1/2 binding was quantified at the identified enhancers. As in pancreas, it was observed two distinct patterns of FOXA1/2 occupancy (FIGS. 7c and 7d) despite similar dynamics in H3K27ac signal (FIGS. 16e and 16f): a subset of class I enhancers exhibited FOXA1/2 occupancy prior to lineage induction (488 class I hepatic enhancers and 368 class I alveolar enhancers), whereas class II enhancers constituted the majority and exhibited de novo FOXA1/2 binding with lineage induction (965 class II hepatic enhancers and 2924 class II alveolar enhancers). These patterns were exemplified by enhancers near hepatic genes Alpha1-Antitrypsin (AA T) and CEBPA (FIG. 7e), as well as lung developmental TF genes SOX2 and NKX2.1 (FIG. 7f).

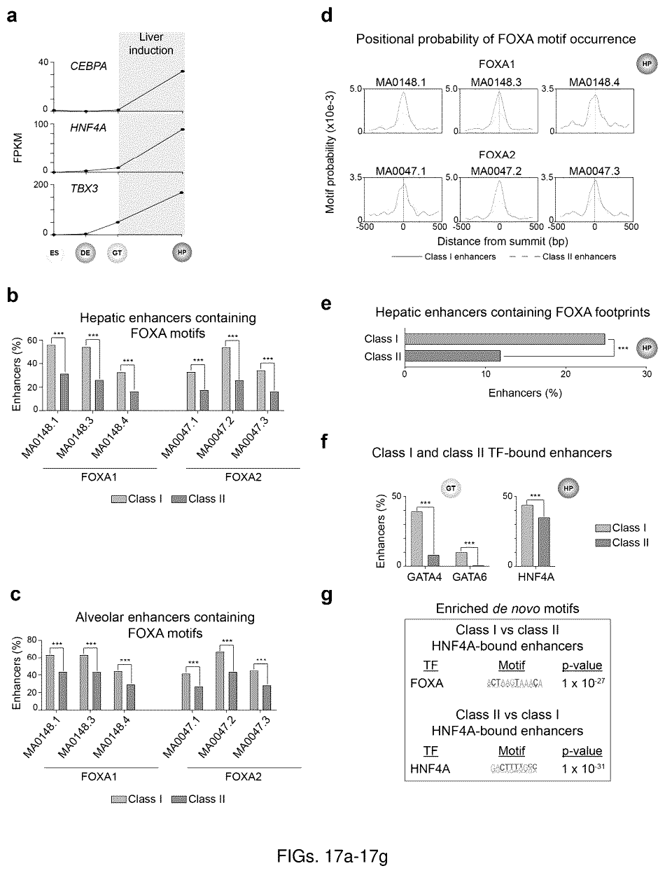

De novo motif analysis at class I against a background of class II hepatic enhancers revealed enrichment for FOXA motifs, GATA motifs, and the motif for the signal-dependent nuclear receptor NR2E1³². Class II enhancers showed comparative enrichment for motifs of the hepatic lineage-determining TFs CEBPA, HNF4A, and TBX^{33, 34}(FIG. 7g), which increased in expression upon liver induction from hESC-GT intermediates (FIG. 17a). FOXA2, HNF4A, and CEBP have been shown to co-bind liver-specific enhancers after liver induction⁷, supporting a potential role for cooperative recruitment of FOXA TFs by these factors. Analogous to the motif enrichment patterns observed in pancreas and liver, alveolar class I enhancers were comparatively enriched for FOXA motifs, GATA motifs, and motifs for signal-dependent TFs NR5A1 (SF1) and TEAD with roles in lung development^{35, 36}, whereas alveolar class II enhancers showed comparative motif enrichment for SOX family TFs and the lung master TF NKX2.1E (FIG. 7h). Thus, as in pancreas, a subset of hepatic and alveolar enhancers with canonical FOXA motifs and enrichment for motifs of signal-dependent TFs are FOXA1/2-bound prior to lineage induction, while de novo FOXA1/2 recruitment occurs at most hepatic and alveolar enhancers upon lineage induction.

To gain further insight into the architecture of hepatic and alveolar enhancers, we examined abundance, strength, and positioning of FOXA motifs. Using the same six FOXA PWMs as for pancreatic enhancers (FIG. 13a), it was observed significant enrichment for occurrence of FOXA motifs at both class I hepatic and class I alveolar enhancers (FIGS. 17b and 17c). It was also found significantly higher log-odds scores for three FOXA PWMs (MA0047.2, MA0148.1, and MA0148.3; P=1.40×10⁻³, 2.00×10⁻³, and 1.60×10⁻², respectively; Wilcoxon rank sum test) at class I compared to class II hepatic enhancers, and two FOXA PWMs (MA0047.3 and MA0148.1; P=3.1×10⁻² and 4.1×10⁻², respectively; Wilcoxon rank sum test) at class I compared to class II alveolar enhancers. Furthermore, FOXA motif occurrence at ATAC-seq summits (444 and 701 ATAC-seq summits in class I and class II enhancers, respectively, at HP stage; FIG. 17d) and occurrence of FOXA footprints (FIG. 17e) were enriched at class I compared to class II hepatic enhancers. Thus, like pancreatic class I enhancers, hepatic and alveolar class I enhancers exhibit sequence features that have been associated with FOXA1/2 pioneering in other contexts^{17, 18}. Moreover, analogous to pancreatic enhancers, we observed preferential binding of GATA4 and GATA6 to class I compared to class II hepatic enhancers at the GT stage (FIG. 17f), but no binding preference of the hepatic lineage-determining TF HNF4A at class II compared to class I hepatic enhancers despite HNF4A motif enrichment at HNF4A-bound class II enhancers (FIGS. 17f and 17g). These results show that similar characteristics of sequence architecture distinguish pancreatic, hepatic, and alveolar class I and class II enhancers.

### Example 10

### Lineage-Specific Recruitment of FOXA1/2 to Unprimed Enhancers

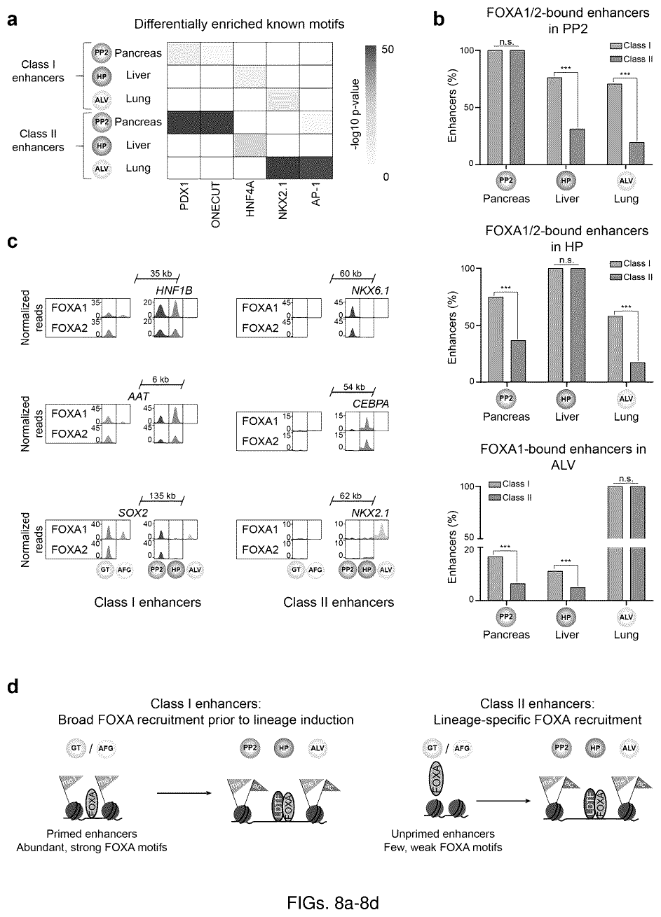

The results suggest a model whereby the full enhancer complement for each endodermal organ lineage is established through (i) FOXA1/2-mediated priming of a small subset of enhancers for each lineage in endodermal precursors prior to lineage induction, and (ii) activation of a larger subset of unprimed enhancers by organ lineage-determining TFs that cooperatively recruit FOXA1/2 upon lineage induction. To determine the relationship between class I and class II enhancers across different endodermal lineages, we performed differential motif enrichment analysis, comparing class I or class II enhancers of each lineage against a background of class I or class II enhancers, respectively, of the alternate lineages. As expected, motifs for lineage-determining TFs for each lineage were enriched at both classes of enhancers. However, motif enrichment was stronger at class II than at class I enhancers (FIG. 8a), lending further support to the model that cooperativity with lineage-determining TFs facilitates lineage-specific FOXA1/2 association with class II enhancers of each organ. Consistent with the binding of FOXA1/2 to class I enhancers in shared developmental precursors prior to lineage induction, it as found that class I enhancers of one organ lineage were more frequently bound by FOXA1/2 in alternate lineages than class II enhancers (FIGS. 8b and 8c). Altogether, these findings support establishment of organ-specific gene expression programs through two distinct mechanisms of FOXA1/2-mediated enhancer activation (FIG. 8d).

### REFERENCES

1. Wang, A. et al. Epigenetic priming of enhancers predicts developmental competence of hESC-derived endodermal lineage intermediates. Cell Stem Cell 16, 386-399 (2015).
2. Zorn, A. M. & Wells, J. M. Vertebrate endoderm development and organ formation. Annu. Rev. Cell Dev. Biol. 25, 221-251 (2009).
3. Lee, K. et al. FOXA2 Is Required for Enhancer Priming during Pancreatic Differentiation. Cell Rep. 28, 382-393 e387 (2019).
4. Genga, R. M. J. et al. Single-cell RNA-sequencing-based CRISPRi screening resolves molecular drivers of early human endoderm development. Cell Rep. 27, 708-718 e710 (2019).
5. Wan, H. et al. Compensatory roles of Foxa1 and Foxa2 during lung morphogenesis. J. Biol. Chem. 280, 13809-13816 (2005).
6. Lee, C. S., Friedman, J. R., Fulmer, J. T. & Kaestner, K. H. The initiation of liver development is dependent on Foxa transcription factors. Nature 435, 944-947 (2005).
7. Iwafuchi-Doi, M. et al. The pioneer transcription factor FoxA maintains an accessible nucleosome configuration at enhancers for tissue-specific gene activation. Mol. Cell 62, 79-91 (2016).
8. Soufi, A. et al. Pioneer transcription factors target partial DNA motifs on nucleosomes to initiate reprogramming. Cell 161, 555-568 (2015).
9. Cirillo, L. A. et al. Opening of compacted chromatin by early developmental transcription factors HNF3 (FoxA) and GATA-4. Mol. Cell 9, 279-289 (2002).
10. Carroll, J. S. et al. Chromosome-wide mapping of estrogen receptor binding reveals long-range regulation requiring the forkhead protein FoxA1. Cell 122, 33-43 (2005).
11. Gualdi, R. et al. Hepatic specification of the gut endoderm in vitro: cell signaling and transcriptional control. Genes Dev. 10, 1670-1682 (1996).
12. Hurtado, A., Holmes, K. A., Ross-Innes, C. S., Schmidt, D. & Carroll, J. S. FOXA1 is a key determinant of estrogen receptor function and endocrine response. Nat. Genet 43, 27-33 (2011).
13. Magnani, L. & Lupien, M. Chromatin and epigenetic determinants of estrogen receptor alpha (ESR1) signaling. Mol. Cell Endocrinol. 382, 633-641 (2014).
14. Wang, Q. et al. Androgen receptor regulates a distinct transcription program in androgen-independent prostate cancer. Cell 138, 245-256 (2009).
15. Lupien, M. et al. FoxA1 translates epigenetic signatures into enhancer-driven lineage-specific transcription. Cell 132, 958-970 (2008).
16. Caizzi, L. et al. Genome-wide activity of unliganded estrogen receptor-alpha in breast cancer cells. Proc. Natl Acad. Sci. USA 111, 4892-4897 (2014).
17. Donaghey, J. et al. Genetic determinants and epigenetic effects of pioneer-factor occupancy. Nat. Genet 50, 250-258 (2018).
18. Swinstead, E. E. et al. Steroid receptors reprogram FoxA1 occupancy through dynamic chromatin transitions. Cell 165, 593-605 (2016).
19. Zhang, Y. et al. GENE REGULATION. Discrete functions of nuclear receptor Rev-erbalpha couple metabolism to the clock. Science 348, 1488-1492 (2015).
20. Paakinaho, V., Swinstead, E. E., Presman, D. M., Grontved, L. & Hager, G. L. Meta-analysis of chromatin programming by steroid receptors. Cell Rep. 28, 3523-3534 e3522 (2019).
21. Whyte, W. A. et al. Master transcription factors and mediator establish super-enhancers at key cell identity genes. Cell 153, 307-319 (2013).
22. Kobberup, S., Nyeng, P., Juhl, K., Hutton, J. & Jensen, J. ETS-family genes in pancreatic development. Dev. Dyn. 236, 3100-3110 (2007).
23. Mamidi, A. et al. Mechano signalling via integrins directs fate decisions of pancreatic progenitors. Nature 564, 114-118 (2018).
24. Huang, W. et al. Retinoic acid plays an evolutionarily conserved and biphasic role in pancreas development. Dev. Biol. 394, 83-93 (2014).
25. Cebola, I. et al. TEAD and YAP regulate the enhancer network of human embryonic pancreatic progenitors. Nat. Cell Biol. 17, 615-626 (2015).
26. Farley, E. K. et al. Suboptimization of developmental enhancers. Science 350, 325-328 (2015).
27. Fornes, O. et al. JASPAR 2020: update of the open-access database of transcription factor binding profiles. Nucleic Acids Res. 48, D87-D92 (2020).
28. Sung, M. H., Guertin, M. J., Baek, S. & Hager, G. L. DNase footprint signatures are dictated by factor dynamics and DNA sequence. Mol. Cell 56, 275-285 (2014).
29. Veres, A. et al. Charting cellular identity during human in vitro beta-cell differentiation. Nature 569, 368-373 (2019).
30. Schaffer, A. E. et al. Nkx6.1 controls a gene regulatory network required for establishing and maintaining pancreatic Beta cell identity. PLoS Genet. 9, e1003274 (2013).
31. Jacob, A. et al. Differentiation of human pluripotent stem cells into functional lung alveolar epithelial cells. Cell Stem Cell 21, 472-488 e410 (2017).
32. Corso-Diaz, X. et al. Co-activator candidate interactions for orphan nuclear receptor NR2E1. BMC Genomics 17, 832 (2016).
33. Kheolamai, P. & Dickson, A. J. Liver-enriched transcription factors are critical for the expression of hepatocyte marker genes in mES-derived hepatocyte-lineage cells. BMC Mol. Biol. 10, 35 (2009).
34. Papaioannou, V. E. The T-box gene family: emerging roles in development, stem cells and cancer. Development 141, 3819-3833 (2014).
35. Ramana, C. V. Insights into the signal transduction pathways of mouse lung type II cells revealed by transcription factor profiling in the transcriptome. Genomics Inf. 17, e8 (2019).
36. Ikonomou, L. et al. The in vivo genetic program of murine primordial lung epithelial progenitors. Nat. Commun. 11, 635 (2020).
37. Herriges, M. & Morrisey, E. E. Lung development: orchestrating the generation and regeneration of a complex organ. Development 141, 502-513 (2014).
38. Zaret, K. S. & Carroll, J. S. Pioneer transcription factors: establishing competence for gene expression. Genes Dev. 25, 2227-2241 (2011).
39. Crocker, J. et al. Low affinity binding site clusters confer hox specificity and regulatory robustness. Cell 160, 191-203 (2015).
40. Li, L. C. et al. Single-cell transcriptomic analyses reveal distinct dorsal/ventral pancreatic programs. EMBO Rep. 19 (2018).
41. Brissova, M. et al. Assessment of human pancreatic islet architecture and composition by laser scanning confocal microscopy. J. Histochem. Cytochem. 53, 1087-1097 (2005).
42. Xie, R. et al. Dynamic chromatin remodeling mediated by polycomb proteins orchestrates pancreatic differentiation of human embryonic stem cells. Cell Stem Cell 12, 224-237 (2013).
43. Schulz, T. C. et al. A scalable system for production of functional pancreatic progenitors from human embryonic stem cells. PLoS ONE 7, e37004 (2012).
44. Rezania, A. et al. Reversal of diabetes with insulin-producing cells derived in vitro from human pluripotent stem cells. Nat. Biotechnol. 32, 1121-1133 (2014).
45. Jin, W. et al. A network of microRNAs acts to promote cell cycle exit and differentiation of human pancreatic endocrine cells. Iscience 21, 681-694 (2019).
46. Hurley, K. et al. Reconstructed single-cell fate trajectories define lineage plasticity windows during differentiation of human PSC-derived distal lung progenitors. Cell Stem Cell 26, 593-608 e598 (2020).
47. Jacob, A. et al. Derivation of self-renewing lung alveolar epithelial type II cells from human pluripotent stem cells. Nat. Protoc. 14, 3303-3332 (2019).
48. Hawkins, F. et al. Prospective isolation of NKX2-1-expressing human lung progenitors derived from pluripotent stem cells. J. Clin. Invest. 127, 2277-2294 (2017).
49. Maddalo, D. et al. In vivo engineering of oncogenic chromosomal rearrangements with the CRISPR/Cas9 system. Nature 516, 423-427 (2014).
50. Ran, F. A. et al. Genome engineering using the CRISPR-Cas9 system. Nat. Protoc. 8, 2281-2308 (2013).
51. Rubinson, D. A. et al. A lentivirus-based system to functionally silence genes in primary mammalian cells, stem cells and transgenic mice by RNA interference. Nat. Genet. 33, 401-406 (2003).
52. Kent, W. J. et al. The human genome browser at UCSC. Genome Res. 12, 996-1006 (2002).
53. Li, H. & Durbin, R. Fast and accurate short read alignment with Burrows-Wheeler transform. Bioinformatics 25, 1754-1760 (2009).
54. Li, H. et al. The sequence alignment/map format and SAMtools. Bioinformatics 25, 2078-2079 (2009).
55. Heinz, S. et al. Simple combinations of lineage-determining transcription factors prime cis-regulatory elements required for macrophage and B cell identities. Mol. Cell 38, 576-589 (2010).
56. Quinlan, A. R. & Hall, I. M. BEDTools: a flexible suite of utilities for comparing genomic features. Bioinformatics 26, 841-842 (2010).
57. Ramirez, F. et al. deepTools2: a next generation web server for deep-sequencing data analysis. Nucleic Acids Res. 44, W160-W165 (2016).
58. Dobin, A. et al. STAR: ultrafast universal RNA-seq aligner. Bioinformatics 29, 15-21 (2013).
59. Trapnell, C. et al. Transcript assembly and quantification by RNA-Seq reveals unannotated transcripts and isoform switching during cell differentiation. Nat. Biotechnol. 28, 511-515 (2010).
60. Love, M. I., Huber, W. & Anders, S. Moderated estimation of fold change and dispersion for RNA-seq data with DESeq2. Genome Biol. 15, 550 (2014).
61. Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat. Methods 10, 1213-1218 (2013).
62. Zhang, Y. et al. Model-based analysis of ChIP-Seq (MACS). Genome Biol. 9, R137 (2008).
63. Geusz, R. J. et al. Pancreatic progenitor epigenome maps prioritize type 2 diabetes risk genes with roles in development. eLife, Research Article (2021).
64. Durand, N. C. et al. Juicebox provides a visualization system for Hi-C contact maps with unlimited zoom. Cell Syst. 3, 99-101 (2016).
65. Rao, S. S. et al. A 3D map of the human genome at kilobase resolution reveals principles of chromatin looping. Cell 159, 1665-1680 (2014).
66. Zheng, G. X. et al. Massively parallel digital transcriptional profiling of single cells. Nat. Commun. 8, 14049 (2017).
67. Stuart, T. et al. Comprehensive Integration of Single-Cell Data. Cell 177, 1888-1902 e1821 (2019).
68. McLean, C. Y. et al. GREAT improves functional interpretation of cis-regulatory regions. Nat. Biotechnol. 28, 495-501 (2010).
69. Zhou, Y. et al. Metascape provides a biologist-oriented resource for the analysis of systems-level datasets. Nat. Commun. 10, 1523 (2019).
70. Loven, J. et al. Selective inhibition of tumor oncogenes by disruption of super-enhancers. Cell 153, 320-334 (2013).
71. Grant, C. E., Bailey, T. L. & Noble, W. S. FIMO: scanning for occurrences of a given motif. Bioinformatics 27, 1017-1018 (2011).
72. Bailey, T. L. & Machanick, P. Inferring direct DNA binding from ChIP-seq. Nucleic Acids Res. 40, e128 (2012).
73. Aylward, A., Chiou, J., Okino, M. L., Kadakia, N. & Gaulton, K. J. Shared genetic risk contributes to type 1 and type 2 diabetes etiology. Hum. Mol. Genet. (2018).
74. Rozowsky, J. et al. AlleleSeq: analysis of allele-specific expression and binding in a network framework. Mol. Syst. Biol. 7, 522 (2011).
75. Mathelier, A. et al. JASPAR 2016: a major expansion and update of the open-access database of transcription factor binding profiles. Nucleic Acids Res. 44, D110-D115 (2016).
76. Consortium, E. P. An integrated encyclopedia of DNA elements in the human genome. Nature 489, 57-74 (2012).
77. Pique-Regi, R. et al. Accurate inference of transcription factor binding from DNA sequence and chromatin accessibility data. Genome Res. 21, 447-455 (2011).
78. Dekker, J. et al. The 4D nucleome project. Nature 549, 219-226 (2017).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A method of improving a yield of insulin-producing cells produced from pluripotent stem cells for transplantation into a patient with diabetes, comprising:
optimizing FOXA binding motifs at an enhancer near the NKX6.1 gene;
acquiring chromatin accessibility across pancreatic progenitors;
broadly expressing the NKX6.1 gene across pancreatic progenitors during development; and
developing beta-cells from pancreatic progenitors to be used for transplantation into the patient with diabetes.

2. The method of claim 1, wherein sequences of the FOXA binding motifs are altered according to CRISPR guides and single stranded oligo donor (SSODN) template.

3. The method of claim 3, wherein a clonal cell line is generated by transfecting stem cells with CRISPR guides and SSODN.

4. The method of claim 3, wherein genotyping of the clonal cell line is commenced.

5. The method of claim 4, wherein clonal cell line containing the altered sequences of the FOXA binding motifs is identified.

6. The method of claim 5, wherein the identified clonal cell line is differentiated to broadly express the NKX6.1 gene at pancreatic progenitor stage.

7. The method of claim 5, wherein the identified clonal cell line is differentiated to show an increased expression of insulin at endocrine progenitor stage.

8. The method of claim 5, wherein the identified clonal cell line is differentiated to show increased beta/enterochromaffin cells than pre-alpha cells at immature beta cell stage.

9. The method of claim 8, wherein the beta/enterochromaffin cells are further differentiated to mature beta cells to be used for transplantation into the patient with diabetes.

10. A method of treating diabetes using the beta cells developed from the method of claim 1.

11. A clonal pluripotent stem cell line for direct differentiation to islet cells, wherein said cell line is generated by transfecting stem cells with CRISPR guides and SSODN.

12. The clonal pluripotent stem cell line of claim 11, wherein said clonal cell line comprises cells containing the altered sequences of the FOXA binding motifs.

13. The clonal pluripotent stem cell line of claim 12, wherein said cell line broadly expresses NKX6.1 gene across cells at pancreatic progenitor cell stage.

14. The clonal pluripotent stem cell line of claim 13, wherein said cell line is differentiated to show an increased expression of insulin at endocrine progenitor stage.

15. The clonal pluripotent stem cell line of claim 14, wherein said cell line is differentiated to show increased beta/enterochromaffin cells than pre-alpha cells at immature beta cell stage.

16. The clonal pluripotent stem cell line of claim 15, wherein the beta/enterochromaffin cells are further differentiated to mature beta cells to be used for transplantation into the patient with diabetes.

17. The clonal pluripotent stem cell line of claim 16, wherein said cell line is a human cell line.
